(19) United States
(12) Patent Application Publication
(10) Pub. No.: US 2007/0238169 A1
(43) Pub. Date: Oct. 11, 2007

(54) Cell sorter and culture system

(75) Inventors: Oscar J. Abilez, Mountain View, CA (US); Peyman Benharash, Los Angeles, CA (US); Christopher K. Zarins, Portola Valley, CA (US)

Correspondence Address: PETERS VERNY , L.L.P., 425 SHERMAN AVENUE, SUITE 230, PALO ALTO, CA, 94306, US

(73) Assignee: The Board of Trustees of the Leland Stanford Junior University

(21) Appl. No.: 11/732,911

(22) Filed: Apr. 5, 2007

Related U.S. Application Data
(60) Provisional application No. 60/791,026, filed on Apr. 11, 2006.

(51) Int. Cl. C12M 3/00; C12N 5/06; C12N 5/08

(52) U.S. Cl. 435/325; 435/289.1; 435/288.7; 435/308.1; 435/366

(57) ABSTRACT

Methods and apparatus are provided for culturing cells under conditions for determining cellular differentiation and for separating cells from culture media based on differentiation. The apparatus comprises a bioreactor, media reservoir, a magnetic cell separator, an inlet port for adding magnetic particles to the bioreactor, and a circulating pump, wherein the bioreactor, media reservoir, magnetic cell separator and inlet port are on a single fluid circuit. The present method and apparatus provides a method for separating cells from culture without removing the cells from culture, so that un-selected cells may be returned to the bioreactor for further culture. The method employs magnetic labeling in culture, where the magnetic label specifically identifies cells to be distinguished, either by separation or retention in the culture. The method and apparatus are further designed to comprise means for electromechanical stimulation of hum embryonic stem cells for preparation of electrically responsive tissue.

### CROSS-REFERENCE TO RELATED APPLICATIONS

This application claims priority from U.S. Provisional Patent Application No. 60/791,026 filed on Apr. 11, 2006, which is hereby incorporated by reference in its entirety.

### STATEMENT OF GOVERNMENTAL SUPPORT

None.

### REFERENCE TO SEQUENCE LISTING

None.

### BACKGROUND OF THE INVENTION

1. Field of the Invention

The present invention relates to the field of cell culture, cell differentiation, and cell separation.

2. Related Art

In the United States in 2002, the prevalence of cardiovascular disease was approximately 70 million and the number of inpatient cardiovascular procedures was approximately 6.8 million.¹ Approximately 1.4 million patients per year undergo procedures requiring arterial cardiovascular grafts.^{2,3} This represents approximately $2.1 billion per year for these procedures (based on the most recent data for average cost per procedure).

Cardiovascular grafts are currently used as bypass grafts, endovascular grafts, and interposition grafts.^{1,3,4} However, the currently available grafts have been limited by variable patency rates, material availability, and immunologic rejection.⁵⁻⁷

In attempts to address these limitations over the last twenty years, experimental human and animal tissue-engineered grafts (TEVG) have been assembled from endothelial cells (EC), smooth muscle cells (SMC), and fibroblast cells (FC)⁸⁻¹²; these experimental TEVG have demonstrated favorable strengths and patency rates. However, their main drawback has been immunologic rejection during in-vivo testing.^{8,10,13}

The creation of a TEVG from autologous stem cells would potentially address these shortcomings, and, furthermore, could potentially serve as the vascular source for other tissue engineered materials such as lung, cardiac, liver, or bone tissue.¹⁴⁻²²

Several stem cell types exist, and one type, the mouse embryonic stem cell (mESC), is well characterized, is readily available, and has no restrictions on its use.²³ Furthermore, groups have reported differentiating mESC into EC and SMC; in addition, FC derived from mouse embryos are commercially available.²⁴⁻²⁹ However, the subsequent in-vitro assembly of these cell types into three-layered blood vessels has not yet been reported. In addition, it is not entirely known how various stimuli affect stem cell differentiation into these cell types.

Furthermore, the differentiation of stem/progenitor cells into myocytes for use in cardiovascular tissue engineering has been ill defined to date. Myocytes must exhibit both functional organization and contractility in order to serve as components for tissue engineered cardiovascular grafts. Recently, groups have demonstrated the salutary effects of electrical stimulation on primary myocyte organization and stem cell differentiation.³⁰⁻³³

Described below are methods and devices for culturing and isolated both differentiated and undifferentiated mammalian (e.g., stem) cells, as well as other cell types. Various levels of chemical and electrical stimulation may be used as part of these methods to allow differentiation of progenitor cells into organized contracting myocytes. In order to test our hypothesis, we applied these stimulation signals to P19 cells, a stem cell line derived from a mouse embryonal carcinoma. Because the P19 cell line is known to have the potential to differentiate into myocytes,³⁴⁻³⁸ this line was used for exemplary experiments described below.

Another aspect of the present invention, which is described below, involves the use of D3 mouse embryonic stem cells that are cultured in a bioreactor, which comprises a pulsatile pump. A three-dimensional culture system may be used in the present apparatus. In such a three-dimensional matrix, cells can grow into multiple layers in 3 dimensions, thereby permitting a longer culture period before confluence. To modulate cell attachment to a substrate, various natural and synthetic substrates have been developed such as those involving short-peptides and sugar-motifs and the like. See “Non-disruptive three-dimensional culture and harvest system for anchorage-dependent cells,” U.S. Pat. No. 6,905,875, hereby incorporated by reference, for further cell culture parameters. The pulsatile conditions mimic physiological conditions and promote differentiation of stem cells. In addition, the pump may be used to move cells and media though the system to a magnetic separation chamber. Mouse embryonic stem cell line D3 is available from ATCC Accession Number CRL −1934.

Publications and Patents

In addition to the references cited at the end of the specification, the following background documents are cited:

Apparatus for exposing cells to pulsatile flow is described in Frangos et al., “Shear Stress Induced Stimulation of Mammalian Cell Metabolism,” Biotechnology and Bioengineering, Vol. 32, Pp. 1053-1060 (1988).

Sodian, et al., “New pulsatile bioreactor for fabrication of tissue-engineered patches,” J Biomed Mater Res. 58(4):401-5(2001) discloses a closed-loop, perfused bioreactor for long-term patch-tissue conditioning, which combines continuous, pulsatile perfusion and mechanical stimulation by periodically stretching the tissue-engineered patch constructs. By adjusting the stroke volume, the stroke rate, and the inspiration/expiration time of the ventilator, it allows various pulsatile flows and different levels of pressure.

Jeonga, et al., “Mechano-active tissue engineering of vascular smooth muscle using pulsatile perfusion bioreactors and elastic PLCL scaffolds,” Biomaterials 26 (2005) 1405-1411, discloses a system in which rabbit aortic smooth muscle cells (SMCs) were seeded onto rubber-like elastic, three-dimensional PLCL [poly(lactide-cocaprolactone), 50:50] scaffolds and subjected to pulsatile strain and shear stress by culturing them in pulsatile perfusion bioreactors for up to 8 weeks.

Bruno et al., U.S. Pat. No. 5,972,721, issued Oct. 26, 1999, discloses a method and apparatus for immunomagnetic separation and concentration of target biological materials in prepared samples (not culture). The overall system combines a reaction subsystem for reacting coated magnetic beads with a sample, a collection subsystem for capturing magnetic beads, a rinsing subsystem for removing debris and a filtering subsystem for removing captured magnetic beads from the collection subsystem.

Terstappen, et al., U.S. Pat. No. 5,993,665, issued Nov. 30, 1999, disclose a method of quantitative analysis of microscopic biological specimens in a fluid medium, in which the specimens are rendered magnetically responsive by immunospecific binding with ferromagnetic colloid. The collected species are resuspended in a second fluid medium, and the relative quantities thereof are enumerated to determine the concentration of the desired biological specimen in the first fluid medium.

Furlong et al., U.S. Pat. No. 6,482,652, issued Nov. 19, 2002 discloses an automated particle sorter that allows the separation of large multicellular biological particles, including embryos, small organisms and the like. The particle sorter provides a means of sorting multicellular aggregates that are too large to be sorted with an electrostatic deflection flow cytometer. A light detection system comprising one or more light detecting elements, e.g., photodiodes, photomultiplier tubes, etc., receives the light and transmits the information to a data processor. The data processor controls a switching mechanism that alters the position of a collection conduit between two set points.

Zborowski and Chalmers, “Magnetic Cell Sorting,” Chapter 12 in Immunochemical Protocols, 3^{rd} Ed., R. Burns, Ed., Humana Press, December 2004, gives examples of commercially available magnetic particles for cell separation. Listed are Dynabeads, BioMag, MACS, BD IMag, Captivate and EasySep.

Sun et al., “Continuous Flow-Through Immunomagnetic Cell Sorting in a Quadrapole Field,” Cytometry 33:469-475 (1998) discloses a flow through magnetic cell separator that was used with human CD4+, CD8+, and CD 45+ cells labeled with mouse anti-human monoclonal antibodies conjugated to FITC and rat anti-mouse antibody conjugated to a colloidal magnetic nanoparticle. Magnets cause labeled cells to move in a radial direction into an outer cylinder for separation.

U.S. Pat. No. 6,890,426, issued May 10, 2005 to Terstappen et al., discloses a magnetic separation apparatus with applications for testing blood incubated with epithelial cell specific ferrofluid in order to isolate tumor cells. A transparent collection wall and a high internal gradient magnetic capture structure are employed.

Chalmers et al., “Flow Through Immunomagnetic Cell Separation,” Biotechnol. Prog. 14:141-148 (1998) disclose a flow through immunomagnetic separation device having a particular magnet design in which a cell suspension, injected in a top port, flows downward with the carrier buffer injected into adjacent ports. Immunomagnetically labeled cells migrate in a cross direction while unlabelled cells are not deflected.

Lara et al., “Enrichment of rare cancer cells through depletion of normal cells using density and flow-through, immunomagnetic separation,” Exp. Hemat. 32:891-904 (2004) discloses a flow-through immunomagnetic cell separation system. The system has quadrapole magnets disposed radially about a channel contained in a core rod, an inlet flow splitter and an outlet flow splitter, radially outwardly displaced form the inlet flow and the core rod.

A protocol for culturing hematopoietic stem cells and hematopoietic progenitor cells is disclosed in U.S. Pat. No. 6,841,386 to Kraus, et al., issued Jan. 11, 2005 and hereby incorporated by reference. It is disclosed there that an endogenous differentiation factor, insulin-like growth factor-1 (IGF-1), interacts with an exogenous anti-differentiation factor that is specific for IGF-1, called insulin-like growth factor binding protein (IGFBP) to affect expansion and differentiation of hematopoietic cells in culture. By modulating the activity of IGF, it is possible to control the differentiation of hematopoietic stem cells and hematopoietic progenitor cells. The protocol described there also uses magnetic separation, in conjunction with a retroviral transduction of cells. Immuno-magnetic selection is done with a lin⁻ cocktail (containing antibodies to CD2, CD3, CD 14, CD16, CD19, CD56, CD66B, and GlyA) added on top of retrovirus infected cells.

U.S. Pat. No. 6,569,654 to Shastri, et al., May 27, 2003, entitled “Electroactive materials for stimulation of biological activity of stem cells,” discloses systems for the stimulation of biological activities within stem cells by applying electromagnetic stimulation to an electroactive material, wherein the electromagnetic stimulation is coupled to the electromagnetic material

U.S. Pat. No. 5,843,741 to Wong, et al., issued Dec. 1, 1998, entitled “Method for altering the differentiation of anchorage dependent cells on an electrically conducting polymer,” discloses a cell culture system for altering the proliferation, differentiation, or function of anchorage dependent cells which includes associating the cells with a surface formed of an electrically conducting polymer and applying an effective amount of a voltage to change the oxidation state of the polymer without damaging the cells.

### BRIEF SUMMARY OF THE INVENTION

The following brief summary is not intended to include all features and aspects of the present invention, nor does it imply that the invention must include all features and aspects discussed in this summary.

The present invention comprises methods and apparatus for culturing and separating cells on the basis of cellular differentiation. The cellular differentiation markers may be any cellular antigenic determinant that may be labeled in culture by a magnetically labeled marker, e.g., antibody. Detailed lists of markers are given below. The present invention further comprises a method and device, including a bioreactor, for culturing undifferentiated cells under defined electromechanical conditions, which result in electrically responsive tissue.

A bioreactor for growing the cells in cell culture media is provided. The bioreactor may have a number of different designs, including supports for anchorage-dependent culture and/or three dimensional cell culture. The bioreactor is preferably configured to operate in continuous, rather than batch mode. A closed fluid circuit, connecting an inlet and an outlet on the bioreactor, is provided for circulation of cells and media and to provide a region for cell separation. Cells and media are circulated from and then to the bioreactor so that the culture process is not disturbed.

The apparatus comprises a pump for pumping media to the bioreactor and for pumping cells and media through the fluid circuit; and an inlet port for introducing magnetic particles into the bioreactor for magnetically labeling cells in culture in the bioreactor. The cells are magnetically labeled while in culture, rather than in a buffer or non-native fluid medium. A magnetic separator, which is preferably located on the fluid circuit, comprises a controllable electromagnet, for separating magnetically labeled cells from circulating media.

In certain embodiments, magnetic separator further comprises a diverter, responsive to the electromagnet, and a collection chamber, attached to the diverter, wherein labeled cells are separated from unlabelled cells on the basis of magnetic labeling, preferably while begin pumped through the fluid circuit, and, again without special separation, re-suspension or rinsing steps.

The separator may be triggered by a separate optical detector, coupled to the magnetic separator, wherein the electromagnet is controlled in response to detection of an optical signal by the optical detector. The optical detector could detect fluorescence from cells that have been dual-labeled with magnets and fluorescent dyes. The optical detector could also be set to be triggered on the basis of size or shape or other properties. A microscope may be used in conjunction with this optical detector, and the cells in the bioreactor may also be examined microscopically.

The bioreactor may further be provided with an electrode that contacts at least a portion of a bioreactor surface adjacent the cultured cells. This electrode may be used to deliver pre-selected pulses of electricity to the cells, so as to cause the cells to adapt into cells having particular electrical activity, e.g., muscle cells. Similarly, the pump used may be a pulsatile pump, which simulates physiological conditions of pumped blood flow, in order to direct cells into certain types of differentiation.

The apparatus may be adapted for certain specific cell culture and isolation of particularly differentiated cells, and, therefore, may be provided as a kit, which may contain cell culture media, stem cells, and growth and differentiation factors intended to derive cells of specific lineages, such as cells to be used in cardiovascular grafts. The differentiated cells are isolated magnetically, with each pass through the circuit yielding additional cells.

### BRIEF DESCRIPTION OF THE DRAWINGS

FIG. 1 shows a general diagram of a cell culture and sorting device according to the present invention;

FIG. 2 shows a custom-made electric cell pulser (2A). The pulser delivered square waves, of various voltage amplitude, pulse width, and pulse frequency, as shown in the diagram below (2B). The electronic circuit design is shown above. The following abbreviations are used in the figure: Op Amp: Operational Amplifier; FET: Field Effect Transistor; VDC: voltage direct current; V+: positive voltage; V−: negative voltage; Sync-OUT: output synchronization from timing chip;

FIG. 3A is a diagram (side view and end view) showing a cell culture bioreactor with an electrode in contact with P19 derived myocytes. 3B is a schematic showing the process design; 3C is a diagram showing a cell culture chamber as in 3A, where the cells are cultured on a flexible tube and comprising an annular electrode;

FIG. 4 is a series of photographs of P19 progenitor cells exposed to both chemical and electrical stimulation, as shown by photos from bioreactors 1-4;

FIG. 5 is a graph showing the number of spontaneously contracting P19-derived myocyte colonies after chemical and electrical stimulation of P19 cells in Bioreactor 1. All cells were exposed to 1% DMSO for five (5) days and to the electrical parameters as listed in the legend;

FIG. 6 is a diagram of a bioreactor layout showing the culture system chamber, circulation loop, and data acquisition system;

FIG. 7 is a schematic of mESC suspended in a basement membrane culture matrix (“culture matrix”) with culture media flowing above the cell suspension. The culture system is secured to the top of the microscope stage.

FIG. 8A shows a schematic side view of one well of the culture system, as illustrated in FIGS. 7, and 8B shows a graph of bead displacement vs. basement membrane culture matrix level; and

FIG. 9 is a diagram of a strategy for assembling a tissue-engineered blood vessel (TEBV).

### DETAILED DESCRIPTION OF THE PREFERRED EMBODIMENT

### I. Overview

1. Introduction

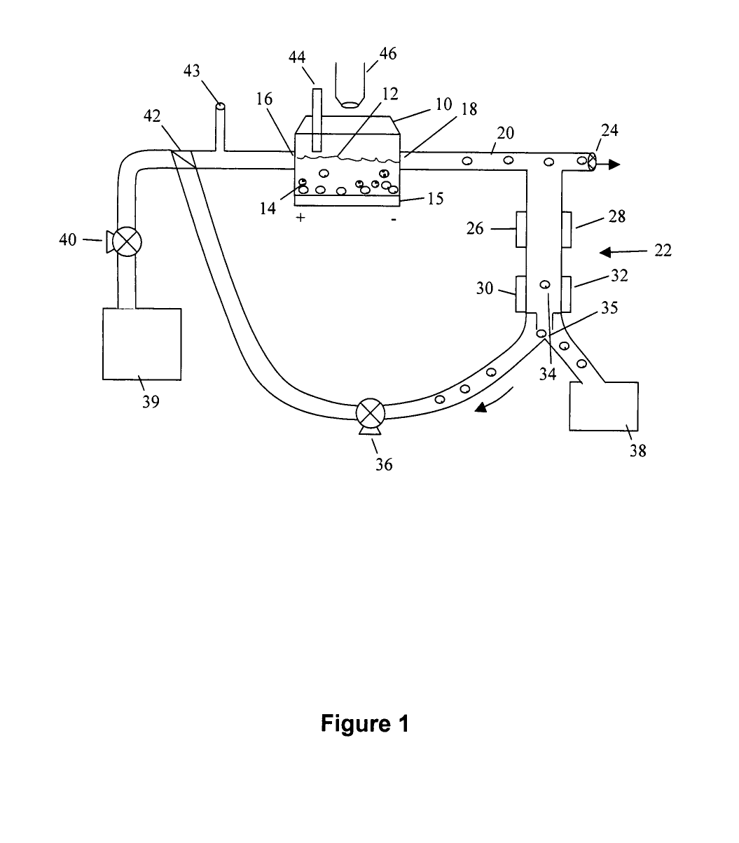

The present methods and apparatus are described in detail in connection with FIG. 1. More detailed descriptions are also provided with regard to certain aspects of the present system. Overall, the present system provides a bioreactor chamber for the culturing of cells, preferably mammalian cells, most preferably stem cells, under defined chemical and physical conditions. The conditions may be chosen for either positive or negative selection, that is, conditions that promote and select for differentiated cells, or conditions that promote and select for undifferentiated (stem) cells. For example, differentiation may be induced by electrical stimuli to the cells. Differentiated or undifferentiated cells are specifically labeled with magnetic beads.

The bioreactor is connected to a pump that pumps media and cells though the system in a closed circuit, and further past a magnetic and/or optical selector. The pump may further be used to create pulsatile, or intermittent, flow, to further mimic physiological conditions. The selector is controlled to bind magnetically labeled cells and then release them into a separate channel in the next flow pulse. This can be done without stopping the cell culture.

The bioreactor is preferably designed for adherent cell culture, i.e., in methylcellulose or Matrigel brand basement membrane material. BD Matrigel™ Matrix is a solubulized basement membrane preparation extracted from EHS mouse sarcoma, a tumor rich in ECM proteins. Its major component is laminin, followed by collagen IV, heparan sulfate proteoglycans, and entactin. At room temperature, BD Matrigel™ Matrix polymerizes to produce biologically active matrix material resembling the mammalian cellular basement membrane. Cells behave as they do in vivo when they are cultured on BD Matrigel™ culture matrix. This provides a physiologically relevant environment for studies of cell morphology, biochemical function, migration or invasion, and gene expression.

Other forms of cell culture may also be used. For example, the cells may be cultured on beads. Under pulsatile conditions, the beads may be made to circulate while the cells are attached. The cells may be cultured in adherent cell culture and then released by gentle trypsinization in order to circulate through the system for circulation.

Further guidance on cell culture systems and design may be found in the following, which are hereby incorporated by reference: U.S. Pat. No. 4,166,768 to Tolbert, et al., issued Sep. 4, 1979, entitled “Continuous cell culture system;” U.S. Pat. No. 4,025,394 to Young issued May 24, 1977, entitled “Fermentation processes using scraped tubular fermentor;” U.S. Pat. No. 4,203,801 to Telling, et al., issued May 20, 1980, entitled “Cell and virus culture systems;” U.S. Pat. No. 5,153,131 to Wolf, et al., issued Oct. 6, 1992, entitled “High aspect reactor vessel and method of use; U.S. Pat. No. 5,518,915 to Naughton, et al., issued May 21, 1996 entitled “Three-Dimensional mucosal cell and tissue culture system;” U.S. Pat. No. 5,580,781, Three-dimensional tumor cell and tissue culture system; U.S. Pat. No. 5,578,485, Three-dimensional blood-brain barrier cell and tissue culture system; U.S. Pat. No. 5,541,107, Three-dimensional bone marrow cell and tissue culture system; U.S. Pat. No. 5,518,915, Three-Dimensional mucosal cell and tissue culture system; U.S. Pat. No. 5,516,681, Three-dimensional pancreatic cell and tissue culture system; U.S. Pat. No. 5,516,680, Three-dimensional kidney cell and tissue culture system; U.S. Pat. No. 5,512,475, Three-dimensional skin cell and tissue culture system; U.S. Pat. No. 5,472,858, Production of recombinant proteins in insect larvae; U.S. Pat. No. 5,443,950, Three-dimensional cell and tissue culture system; U.S. Pat. No. 5,266,480 Three-dimensional skin culture system; U.S. Pat. No. 5,160,490, Three-dimensional cell and tissue culture apparatus; and other designs.

2. Electrical Stimuli to Promote Controlled Differentiation

For years, chemical and electrical stimuli have been noted in the early embryo.³⁹ The effects of electrical stimulation on myocyte organization³⁰⁻³² and stem cell differentiation³³ have recently been described. The work of Radisic, et al, demonstrated that myocytes exhibit structural, ultra-structural, and functional changes upon prolonged electrical stimulation. However, the goal of their work was to demonstrate these changes in primary myocytes and not in progenitor-derived myocytes. Also, in light of Deisseroth's description of neuronal stem cell differentiation with electrical stimulation, our results expand on the use of electrical stimulation on stem cells to derive myocytes.

Creating a layer of myocytes with architectural and electrical organization is a critical step towards production of functional engineered cardiovascular grafts. The application of chemical and electrical signals to a multi-dimensional scaffold and assembly of different cell types may serve to generate more of a physiologic cardiovascular organization.

In this application, the cells in the bioreactor are differentiated, and the sorter is used to remove undifferentiated cells, which would not be appropriate for a graft, due to the risk of teratoma or other irregular growth inside the host.

Thus, synchronization of stem cell-derived myocytes using external pacing is one preferred embodiment of the present system. The ability to synchronize multiple colonies with an external field yields insights into the electrophysiological response of these myocytes. Long term synchronization, could lead to beneficial effects with regards to cell-cell communication and structural and ultra-structural organization as suggested by the work of Radisic et al.

Altering the rate of the synchronization signal may allow generation of myocytes with more of a smooth muscle phenotype through differential expression of various types of ion channels. This will also need to be investigated in future studies.

3. Mechanical (Pulsatile) and Other Stimulation

Mechanical forces have been shown to affect organization of cell cultures and directly influence blood vessel physiology.⁴⁰⁻⁴⁴ Combining these effects with chemical and electrical stimulation will ultimately provide a more realistic niche for stem cell differentiation and organization.

A by-product of electrical stimulation appears to be generation of free-radicals through hydrolysis. Application of flow to cell cultures under electrical stimulation may not only aid in cellular organization, but would also mitigate the deleterious effects of free-radicals by continuously removing them from the local environment.

Clearly, manipulation of other stimuli such as oxygen tension, pH, concentration of growth factors, such as vascular-endothelial growth factor (VEGF) and transforming growth factor-beta (TGF-β), will influence differentiation and subsequent proliferation of stem cells. These stimuli, which have been studied individually in great detail,⁴⁵⁻⁴⁷ may be designed to function in combination with mechanical, electrical, and other chemical stimuli.

4. Cell Culture Conditions

Annexin-V immunocytochemistry and propidium iodide staining to quantify degrees of apoptosis and necrosis, respectively, may be employed to verify cell viability. The examples below used a mixed population of undifferentiated and differentiated P19 cells prior to exposing them to the chemical and electrical stimulation. The presence of already differentiated cells probably led to overall lower yields of differentiated myocytes; however, this may be resolved with different starting cells.

5. Bioreactor

Several bioreactors and culture systems have been described for cardiovascular tissue engineering. However, most have been designed to culture and condition primary cells (endothelial cells (EC), smooth muscle cells (SMC), and fibroblast cells (FC)) that have an already differentiated phenotype. None have been designed specifically to condition and stimulate stem cells in 3D and from their most pluripotent state into other cell types such as EC, SMC, and FC.

The present bioreactor may allow for placement of embryoid bodies, an aggregate of pluripotent stem cells, in a 3D matrix with concurrent exposure to fully adjustable shear, flow, and pressure, in addition to pH, oxygen, VEGF, and other soluble factors. Embryoid bodies may be formed as tissue-like spheroids in suspension culture. Human and mouse embryonic stem cell lines require aggregation of multiple ES lines to efficiently initiate embryoid body formation.

These stimuli, either independently or in limited combinations, have been shown to affect cellular proliferation, differentiation, and apoptosis. Early exposure to hemodynamic and chemical stimuli is a critical step for simulating in-vivo conditions in organogenesis. It is likely that an embryoid body is exposed to very different stimuli as compared to a fully differentiated cell (e.g., endothelial cell in an adult aorta). With this in mind, we have designed our bioreactor to allow for application of a full spectrum of temporal and spatial stimuli in various phases of differentiation. It is further understood that key parameters such as pulsatile flow, culture conditions, electrical stimulation, injection of growth factors and the like may be computer controlled according to a specific protocol developed to yield a uniform selected population.

At different levels of the Matrigel™ basement membrane culture matrix layer in our culture system, there were varying degrees of bead displacement. This gradient of movement may allow us to visualize differential changes of cell behavior in response to mechanical stimuli. Overall, the trend observed for bead displacement (which is a model for cell displacement) agrees with the results expected from a deformable structure with one fixed surface and one free surface exposed to flow. At the interface between the chamber bottom and the Matrigel™ basement membrane culture matrix layer (Level 0) of the culture system, the maximum bead displacement for each flow was essentially zero (0). At increasing Matrigel™ basement membrane culture matrix levels (Levels 1-5), the maximum bead displacement within each level increased. At Level 5, near the interface between the culture matrix layer and fluid flow, the maximum displacement for each flow reached its largest value of approximately 20× bead diameters or 120 μm.

Although the average distance between levels was 400 μm, there was some variability in their specific inter-level distance; i.e., the closer the levels were together, the less the difference in their displacements. A less significant factor was the inhomogeneous solidification of Culture matrix, leading to areas with different deformabilities. Finally, in analyzing the displacements, there is a possibility that frames showing maximum displacement may not have been captured due to the finite capture rate of the CCD camera (30 frames/sec).

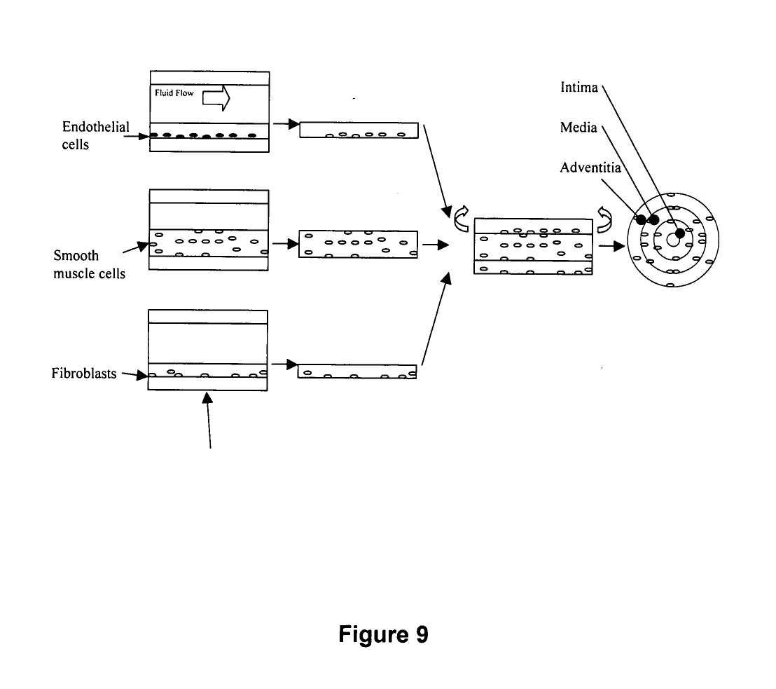

The gradient of bead displacement within the Matrigel™ basement membrane culture matrix will allow for assessing various magnitudes of shear on differentiation of embedded stem cells at different layers of the Culture matrix. If the displacement at a given layer produces the optimal cell type and alignment, then the cells can be selectively embedded only in that layer. However, it is likely that a gradient of displacement is present within the vascular wall, as has been demonstrated by others; if this is the case, then our present configuration will mimic physiologic conditions more accurately. One of the inherent advantages of having cells in a 3D matrix is the capability to create multiple layers of the same or different cell types and then assemble these cell layers into a more complex structure such as a blood vessel (see FIG. 9, described below). Similar techniques for assembling 2D layers have previously been described. (L'Heureux, N., Paquet, S., Labbe, R., Germain, L., and Auger, F. A. A completely biological tissue-engineered human blood vessel. Faseb Journal 12: 47-56, 1998. Ito, A., Ino, K., Hayashida, M., Kobayashi, T., Matsunuma, H., Kagami, H., Ueda, M., and Honda, H. Novel Methodology for Fabrication of Tissue-Engineered Tubular Constructs Using Magnetite Nanoparticles and Magnetic Force. Tissue Engineering 11: 1553-1561, 2005.)

Determination of viability and characteristics of cells that remain in the basement membrane culture matrix and of the cells that wash away is very important. The cells that remain in the basement membrane culture matrix will remain viable as this has been established in other studies of 3D culture. We also expect that the cells that remain in basement membrane culture matrix will respond to mechanical stimuli differently, due to their exposure to shear, than the ones that are washed away. This expectation is based on the well-described response of attached cells in a 2D layer to mechanical stimuli.

The WSS (wall shear stress) that resulted from our flow rates described below compared to the WSS expected in the adult mouse aorta. However, the present system will allows for changes to the culture chamber geometry, circulating fluid flow rate, and circulating fluid viscosity in order to increase the WSS accordingly. The ability to finely tune the WSS is an advantage because low WSS may be initially necessary for stem cell incorporation into the Matrigel™ basement membrane culture matrix and for extracellular matrix production.

### II. Generalized Method and Apparatus

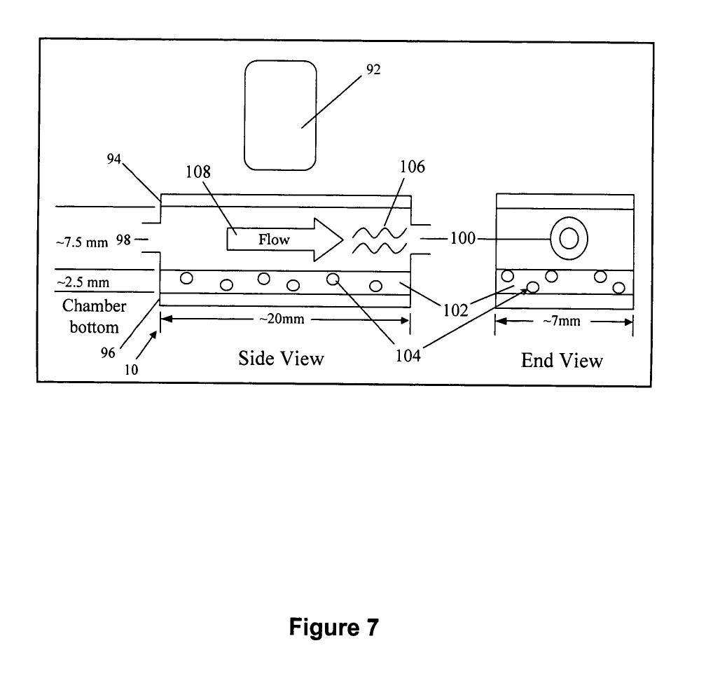

Referring now to FIG. 1, a bioreactor chamber 10 is shown which contains media 12 and cells 14, which may include both stem cells and feeder cells. An electrode 15, as shown in other figures below, may also be on the bottom of the bioreactor to provide stimulation for differentiation into a myocyte lineage. Also, as shown in more detail in FIG. 7, an inlet port 16 and outlet port 18 connect a circulating path 20 for cells and media to circulate to and from the bioreactor. As shown, the circulating path 20 takes cells and media from the bioreactor to a detector and separator area 22. A filter 24 may be provided just downstream from the outlet 18 to remove cellular debris, undissolved media, etc. The cell separation area 22 comprises a magnetic component and an optional optical component. The optical component preferably comprises a laser 26 and an optical detector 28 (e.g., photodiode, CCD detector or photomultiplier tube mounted on a microscope) for detecting the presence of a fluorescent label on a cell illuminated by the laser. The optical sensor is used to trigger magnetic separation. Image feature analysis (such as size, color, shape, etc) could be detected and the specific morphologic feature could trigger the magnetic separator. An electromagnet is coupled to the optical detector and comprises one or a pair or opposed electromagnets 30, 32, in a narrow channel in the circulating path 20. The magnets are only triggered when an optical signal is detected by fluorescence of a labeled antibody, as described below.

The electromagnet(s) are magnetized to bind paramagnetically labeled cells 34, labeled, e.g., with BD IMag particles, flowing in the circulating path 20, from the bioreactor chamber 10. Downstream of the electromagnets 30, 32 is a diverter 35 which separates labeled cells from unlabelled cells if optical detection and/or a continuous flow mode is not used. The diverter may comprise a changeable valve, movable in response to release of cells from the electromagnets 30, 32; or it may operate in a continuous mode if the electromagnets are adjusted to divert the flow of media and cells towards one side of the channel, rather than completely binding the cells. Alternatively, as described below, diversion may be accomplished purely by magnetic forces, without the need for a separate valve.

In continuous mode, magnet 30 is normally on, and magnet 32 is normally off, when there is no fluorescent signal. This will divert cells into the circulating path 20 and away from the collection vessel 38. Upon detection of a signal, a trigger pulse is sent to a timing circuit which accounts for the particle speed and drag between the optical detector and the magnetic array. At the time that the fluorescent cell(s) reach(s) the magnets, magnet 30 is turned off and magnet 32 is turned on, causing the flow to divert towards collection vessel 38. The collection vessel 38 may also be provided with an electromagnet to attract labeled cells at the appropriate time. The collection vessel may be maintained so that the collected cells are still viable and suitable for further culture and/or in vivo growth.

Cells are either diverted to the collection vessel 38 for further cell processing or discarded, or to a continuation of the circulating path 20. A pump 36 acts to circulate cells from the diverter back towards the media container and the bioreactor chamber 10. As described in detail below, the pump may be operated in a pulsed mode to simulate physiological conditions, such as arterial flow. Fresh media from the media chamber 39 is pumped by pump 40, through a valve 42 for controlling the flow of the circulating media. This valve may be located at any point on the circuit, but is preferably just upstream from the media chamber. In addition, in the vicinity of the bioreactor chamber, an inlet port 43 is provided to allow sterile injection of paramagnetic beads, antibodies or other reagents that will be incubated in the bioreactor chamber with the cells cultured there. A monitor inlet 44 extending from the environment directly into the chamber may also be used, and further provides monitoring of temperature, pO₂, pCO₂, pH, temperature, and other cell culture conditions. As described below, a microscope is positioned to observe cells and tissue organization in the bioreactor chamber 10.

Thus, in operation, the present culture system is completely isolated from the environment. Stem cells 14 and media are introduced into the bioreactor chamber 10 through an enclosed system and cultured under differentiating or non-differentiating conditions. Under differentiating conditions, they may be stimulated electrically or mechanically (pulsatile flow). Appropriate growth factors are administered. The cells are then incubated with paramagnetic beads attached to antibodies for specific markers, either of non-differentiation or differentiation. Cells to be selected may be removed from the substrate by trypsinization, as is known in the field of cell culture. Labeled, loose cells are pumped to the sorter, where the labeled cells are isolated for further processing or discarded.

In the case of tissue engineering, the tissue from the bioreactor 10 is harvested as differentiated fibroblast, myocyte, and endothelial layers, and assembled, as described further below. In the case of individual cell isolation, e.g., stem cells, repeated passes of labeled stem cells are carried out and the stem cell population is accumulated in the collection vessel 38.

As can be seen, the cells are labeled, selected and separated, all in their original media. In an alternative embodiment, the magnetic separator 30, 32 is integral to the bioreactor chamber 10, thus allowing for ‘in-situ’ separation. For example, adherent cells could be magnetically labeled and then trypsinized in a given chamber. Then, in the same chamber, an electromagnet could be turned on. Next, flow could then be turned on to wash away the non-magnetically captured cells. Finally, the cells could be allowed to re-attach and then be exposed to various stimuli.

Appropriate labels and protocols may be designed depending on the labels to be used and the electromagnet design. In some cases, the paramagnetic beads may be used to label all cells, and activated by fluorescence from a selective (antibody label) detected by optical detector 28.

Streptavidin-coated paramagnetic beads (2.8 μm diameter, M-280) beads may be obtained from Dynal Corp. in Lake Success, N.Y. Streptavidin-coated colloidal ferrofluid magnetic particles, or “MACS”, beads may be obtained from Miltenyi Biotec Corp. in Auburn, Calif.

By using streptavidin-coated beads, one may specifically attach these beads to biotin-labeled antibodies or other cell type specific proteins. As an example of this implementation, one may refer to the presently marketed BD IMag™ Cell Separation System. This system utilizes magnetic bead technology for enrichment or depletion of specific cell populations in a prepared sample. BD Biosciences Pharmingen provides antibody-labeled magnetic particles for enrichment or depletion of leukocyte subpopulations. Similar particles may be prepared for stem cell markers.

BD IMag particles range in size between 0.1 and 0.45 μm and are coated with BD Pharmingen monoclonal antibodies. These particles are optimized for positive or negative selection of leukocyte subpopulations using either the BD IMagnet™ direct magnet or a magnetic separation column. BD IMag particles coated with specific monoclonal antibodies are added to a cell suspension. The BD IMag particles will specifically bind to the subpopulation of interest. The labeled cell suspension can then be placed in the magnetic field of the BD IMagnet direct magnet, or alternatively, the cells can be run over a separation column that has been placed in a magnetic field. Captured cells can be run on a flow cytometer with the BD IMag particles intact.

When embryoid bodies are grown in suspension culture in vitro, they undergo only a limited amount of morphological development. When these same embryoid bodies are permitted to attach to the surface of a culture dish, a wide variety of new morphological cell types appear.

A protocol for the culture of stem cells into cardiomyocytes is described in Shmelkov et al., “Cytokine Preconditioning Promotes Codifferentiation of Human Fetal Liver CD133+ Stem Cells Into Angiomyogenic Tissue,” (Circulation, 2005; 111:1175-1183.) This publication discloses that human fetal liver CD133+ and CD133− cell subpopulations were cultured with 5′-azacytidine or vascular endothelial growth factor (VEGF165) and/or brain-derived nerve growth factor (BDNF). CD133+ but not CD133− cells from human fetal liver codifferentiated into spindle-shaped cells, as well as flat adherent multinucleated cells capable of spontaneous contractions in culture. The resulting spindle-shaped cells were confirmed to be endothelial cells by immunohistochemistry analysis for von Willebrand factor and by acetylated LDL uptake. Multinucleated cells were characterized as striated muscles by electron microscopy and immunohistochemistry analysis for myosin heavy chain. Presence of VEGF165 and BDNF significantly enhanced angiomyogenesis in vitro. Inoculation of cells derived from CD133⁺ cells, but not CD133⁻ cells, into the ear pinna of NOD/SCID mice resulted in the formation of cardiomyocytes, as identified by immunostaining with cardiac troponin-T antibody. These cells generated electrical action potentials, detectable by ECG tracing.

Described below are exemplary differentiation reagents and markers, which may be used in order to measure the differentiated state of the cells under culture and to select cells for labeling and removal from the culture system. These markers are summarized as stem cell markers; undifferentiated markers; and endothelial cell (EC) and smooth muscle cell (SMC) progenitor markers.

Stem cell markers: CD34⁺, Thy⁺, Lin⁻, CD2⁻, CD3⁻, CD4⁻, CD8⁻, CD10⁻, CD14⁻, CD15⁻, CD19⁻, CD20⁻, CD33⁻, CD34⁻, CD381^{o/-}, CD45RA⁻, CD 59^{+/-}, CD71⁻, CDW109⁺, glycophorin⁻, AC133⁺, HLA⁻DR^{+/-}, c-kit⁺, and EM⁺. Lin⁻ refers to a cell population selected on the basis of lack of expression of at least one lineage specific marker, for example CD2, CD3, CD14, and CD56. Further description is found in US PGPUB 2004/0241856 by Cooke, published Dec. 2, 2004, entitled “Methods and compositions for modulating stem cells,” hereby incorporated by reference.

Undifferentiated markers: SSEA-1 antibody: SSEA-1 is a carbohydrate epitope associated with cell adhesion, migration and differentiation. Expression of SSEA-1 is down regulated following differentiation of murine EC and ES cells. In contrast, the differentiation of human EC and ES cells is characterized by an increase in SSEA-1 expression. Alkaline phosphatase: Undifferentiated human Embryonal Carcinoma and Embryonic Stem cells have been shown to express very high levels of Alkaline Phosphatase isozyme that is indistinguishable from the isozyme found in liver, bone and kidney. Expression levels of AP decrease following stem cell differentiation. Oct-4: The POU transcription factor Oct4, expressed in ESCs and germ cells, is strongly implicated in the process of maintaining as well as regaining stem-cell pluripotency and functions as a key regulator of mammalian germline development.

As described in Henderson et al., “Preimplantation Human Embryos and Embryonic Stem Cells Show Comparable Expression of Stage-Specific Embryonic Antigens,” Stem Cells, 2002; 20:329-337, hereby incorporated by reference for further reference to stem cell markers, the glycolipid antigens with globoseries carbohydrate core structures, SSEA3 and SSEA4, are expressed by unfertilized eggs and early cleavage embryos, but disappear by the blastocyst stage and are not expressed by cells of the ICM (inner cell mass); these antigens are expressed by the primitive endoderm. Likewise, murine ES cells also do not express either SSEA3 or SSEA4. In culture, the differentiation of murine EC and ES cells is typically characterized by the loss of SSEA1 expression and may be accompanied, in some instances, by the appearance of SSEA3 and SSEA4.

By contrast, human EC cells typically express SSEA3 and SSEA4 but not SSEA1, while their differentiation is characterized by the downregulation of SSEA3 and SSEA4 and upregulation of SSEA1. The initial reports of hES cell lines have indicated that they too express SSEA3 and SSEA4, as well as the keratan sulphate-associated antigens, TRA-1-60 and TRA-1-81, which are also characteristic of human EC cells. The above cited paper further discloses that hES cells in culture and the ICM cells from human blastocysts share expression of SSEA3, SSEA4, TRA-1-60, and TRA-1-81 and do not express SSEA1.

Endothelial (EC)/Smooth muscle cell (SMC) progenitor markers: Flk1. Expression of the VEGF receptor Flk1 (VEGFR-2) has been used extensively to define the vascular and hematovascular lineages. Further description may be found in Blood, 1 Jan. 2006, Vol. 107, No. 1, pp. 3-4, hereby incorporated by reference.

Another marker, Oct4 expression becomes restricted to the inner cell mass and epiblast. After gastrulation Oct4 is active only in germ cells and is silent in somatic cells

EC markers: CD31 is constitutively expressed on the surface of endothelial cells, and concentrated at the junction between them. It is also weakly expressed on many peripheral lymphoid cells and platelets. CD31 interacts homotypically in cell adhesion assays.

SMC markers: Actin is detected by an antibody monoclonal antibody, which is specific for the alpha smooth muscle actin isoform. Calponin-h1 is a 34-kDa myofibrillar thin filament, actin-binding protein that is expressed exclusively in smooth muscle cells (SMCs) in adult animals. During murine embryonic development, calponin-h1 gene expression is (i) detectable in E9.5 embryos in the dorsal aorta, cardiac outflow tract, and tubular heart, (ii) sequentially up-regulated in SMC-containing tissues, and (iii) down-regulated to non-detectable levels in the heart during late fetal development. SM myosin heavy chain reactivity is first seen in the trachea and bronchi of saccular lung at the time of birth, when other SMMHC isoforms also are present. Immunoreactivity spreads distally through the airways as development proceeds, reaching the level of alveolar septae in the adult.

In order to maintain an undifferentiated state or to induce terminal differentiation, a number of biological factors may be used. Exemplary factors are listed below.

Growth and differentiation factors: LIF “leukemia inhibitory factor” regulates ex vivo stem cell proliferation. Addition of LIF to stem cell culture media initially reduces the number of differentiating cells, although the undifferentiated stem-cell population declines with successive passaging in the presence of LIF alone. BMPs are known to antagonize neural differentiation, so one may also add Bmp2 or Bmp4 to LIF-containing ES cultures. LIF plus Bmp may be used to maintain pure populations of undifferentiated, diploid ES cells even after extended passage.

Embryonic hemangioblasts are characterized by expression of the vascular endothelial cell growth factor receptor-2, VEGFR-2, and have high proliferative potential with blast colony formation in response to VEGF. The earliest precursor of both hematopoietic and endothelial cell lineage are thought to have diverged from embryonic ventral endothelium, which has been shown to express VEGF receptors as well as GATA-2 and alpha4-integrins. Subsequent to capillary tube formation, the newly created vasculogenic vessels undergo sprouting, tapering, remodeling, and regression under the direction of VEGF, angiopoietins, and other factors, a process termed angiogenesis.

PDGF-BB, Recombinant Human Platelet-Derived Growth Factor-BB, dramatically reduces smooth muscle (SM) alpha-actin synthesis. See Holycross, et al., “Platelet-derived growth factor-BB-induced suppression of smooth muscle cell differentia,” Circulation Research, Vol 71, 1525-1532, (1992), hereby incorporated by reference.

Dibutyryl-cyclicAMP, isoprotemol or N6,O2′-dibutyryl adenosine 3′:5′-monophosphate (dibutyryl cyclic AMP), cyclic AMP not only controls the synthesis of DNA by epidermal cells in culture but also induces the process of differentiation toward keratinization. It has also been reported to induce differentiation, along with retinoic acid, of smooth muscle.

Retinoic acid induces stem cell differentiation into keratin, glial fibrillary acid protein, and neurofilament-positive somatic cells. The differentiation is associated with the disappearance of oligosaccharide surface antigens typical of the undifferentiated stem cells; a loss of proteins typical of undifferentiated cells and the appearance of new proteins; and the deposition of extracellular matrix.

Other known factors may be used. See Takahashi et al., “Ascorbic Acid Enhances Differentiation of Embryonic Stem Cells Into Cardiac Myocytes,” Circulation, 2003; 107:1912.

In general, it is understood that a protocol for a chosen differentiation culture must involve a coordinated regimen of several factors. Various cell culture protocols for stem cell differentiation are known and may be adapted to the present method, given the details presented here.

### III. EXAMPLES

### Example 1

### Electric Cell Pulser and Bioreactor

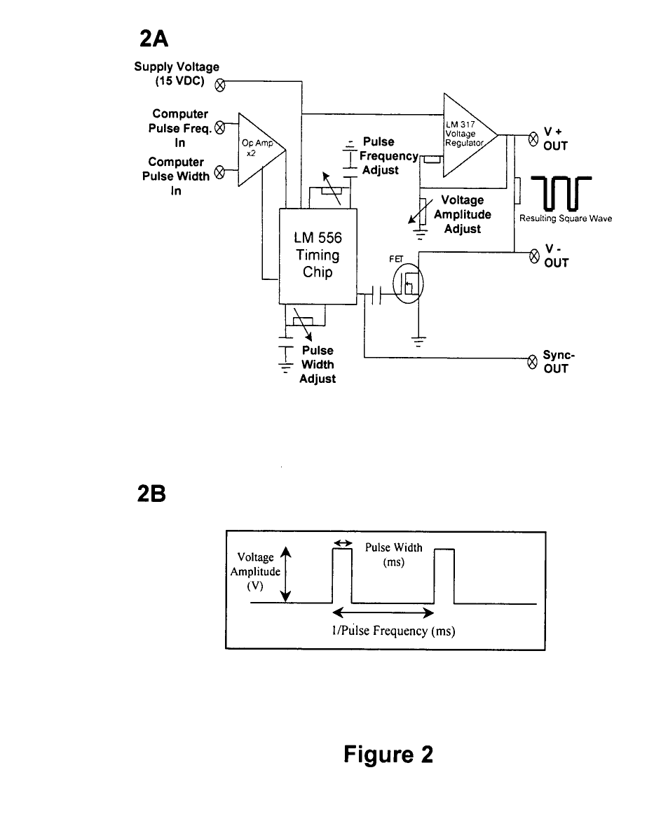

A custom-made cell pulser was made to electrically stimulate the P 19 cells. The electric cell pulser, its output pulse characteristics, and its electronic design is shown in FIG. 2A.

The electric cell pulser was designed with four (4) channels to simultaneously stimulate cells in four (4) separate bioreactors. Each channel could deliver a square wave pulse of varying voltage amplitude (1-10 V), width (0.5-125 ms), and frequency (0.6-300 Hz). Due to technical limitations (which have since been addressed), the minimum frequency we could obtain for our experiments was 10 Hz. The electronic circuit design of the cell pulser is shown in FIG. 2A and the amplitude, pulse width and frequency parameters are shown in FIG. 2B. We implemented an LM 556 timing chip (Jameco Electronics, Belmont, Calif.) to coordinate the manual pulse width and frequency adjustment. This chip also allowed computer control of the pulse width and frequency via two (2) operational amplifiers (Op Amp) (Jameco Electronics, Belmont, Calif.). The voltage amplitude adjustment was achieved with an LM 317 voltage regulator (Jameco Electronics, Belmont, Calif.). A field effect transistor (FET) (Jameco Electronics, Belmont, Calif.) was used in an open collector configuration. A triple output power supply (Tektronix Model CPS 250, Beaverton, Oreg.) was used to provide fifteen-volt direct current (15 VDC) to both the timing chip and voltage regulator. Finally, to facilitate the observation of the output from the timing chip on our digital storage oscilloscope (Hitachi Model VC-6025, Tokyo, Japan), we implemented a synchronization (sync-OUT) channel.

Bioreactor

To assemble the four individual bioreactors, which were placed in an incubator, we first obtained individual off-the shelf items. We obtained a four-well Lab-Tek™ Chamber-Slide system (Nalge Nunc # 177437, Rochester, N.Y.). This chamber-slide system uses a chamber made of polypropylene and a slide made of Permanox™.

We then used a standard drill press fitted with a 1/64″ drill bit to drill one hole at each end of each well (eight (8) total holes were made). Into each hole we placed approximately 1 cm of 99% pure gold wire (Sigma-Aldrich, St. Louis Mo.) to serve as the electrodes for electrical stimulation. The outside ends of the gold electrodes were connected to flat ribbon computer wire (Jameco Electronics, Belmont, Calif.) via gold plated connectors (Jameco Electronics, Belmont, Calif.). (Finally, we used Loctite™ Five-Minute epoxy (Loctite-Henkel, Rocky Hill, Conn.) to attach the gold electrodes to the chamber to obtain the completed bioreactor comprising four adjacent chambers. The distance between the gold electrodes was one (1) cm. Applied voltages from the electric cell pulser, described previously, were divided by this distance to obtain field strengths in V/cm.

For all chemical and electrical stimulation experiments, four bioreactors were used. The bioreactors were placed in a 37° C., 5% CO₂ incubator and were connected to the electric cell pulser and power supply. A data acquisition system was used to control the pulse width and frequency of the electric cell pulser. Our system consisted of National Instruments cFP-2000 control module hardware and National Instruments LabView 7.1 software (National Instruments, Austin, Tex.). The hardware was directly connected to the cell pulser via Bayonet Nut Coupling (BNC) connectors.

Finally, the microscope used to observe the daily activity in the bioreactors was a Leica DM-IL (Leica Microsystems USA, Bannockburn, Ill.) inverted microscope fitted with 10× oculars, and 4×, 10×, 20×, and 40× objectives; this combination of optics allowed magnification of 40×, 100×, 200×, and 400×, respectively. Attached to the microscope was a Retiga 2000R high-speed digital CCD camera (QImaging, Burnaby, BC, Canada) capable of taking single frames and/or video-quality movies (30 frames/sec).

### Example 2

### Complete Media for P19 Cell Culture

In order to perform cell culture, we prepared complete media as follows. The media consisted of alpha-MEM with ribonucleosides and deoxynucleosides (α-MEM) (Invitrogen # 12571-063, Carlsbad, Calif.) supplemented with 7.5% Calf Bovine Serum (CBS) (American Type Culture Collection, ATCC #30-2030, Manassas, Va.) and 2.5% Fetal Bovine Serum (FBS) (GIBCO #26140-079, Carlsbad, Calif.). Next, to the above mixture, penicillin-streptomycin (PS) (GIBCO #15140-122, Carlsbad, Calif.) was added (diluted from a 100× concentration of stock solution to a final concentration of 1× in the complete media). Finally, beta-mercaptoethanol (β-ME) was added to a final concentration of 0.1 mM.

The formulations may be summarized as follows:

TABLE 1

| | Amount | Vendor |
|---|---|---|
| Reagent | | |
| P19 Cells | 1 mL Vial | ATCC |
| α-MEM (w/riboNS & deoxyNS) | Balance | Gibco/Biostores |
| Calf Bovine Serum | 7.5% | ATCC |
| Fetal Bovine Serum, US Qual | 2.5% | Gibco/Biostores |
| Penicillin/Streptomycin (100×) | 1× | Gibco/Biostores |
| Sodium Bicarbonate, 7.5% | 1.5 g/L | Gibco/Biostores |
| β-ME | 0.1 mM | Gibco/Biostores |
| Total | | |
| CO₂ | 5.0% | Praxair |
| To Differentiate Into Myocytes | | |
| Dimethylsulfoxide (DMSO) | 0.5-1.0% | Sigma |
| Complete Media (see above) | 99.5-99% | N/A |
| Freezing Media | | |
| DMSO | 5% (v/v) | Sigma |
| Complete Media (see above) | 95% (v/v) | N/A |

### Example 3

### P19 Cell Culture

In order to perform cell culture, we first obtained a 1 mL vial of frozen P19 mouse embryonal carcinoma stem cells (P19 cells) (ATCC # CRL-1825, Manassas, Va.). The vial of cells was thawed in a 37° C. water bath and the cells were then re-suspended in 9 mL of new complete media in a 15 mL tube. The tube was then spun down in a VWR Clinical 200 centrifuge (VWR #82013-812, West Chester, Pa.) at 300×g (corresponding to 1750 revolutions per minute (rpm) based on the size of the centrifuge rotor) for 3 minutes. The media was then aspirated while the pellet of cells was left in the tube. Next, 5 mL of new fresh complete media was added to the tube. The clump of cells was then dissociated by pipetting up and down. The dissociated cells and new media were then transferred into a T-25 tissue-culture grade flask (Becton Dickinson Biosciences # 353108, Bedford, Mass.). The flask containing the cells was then placed in a 37° C., 5% CO₂ incubator (Fisher Scientific Isotemp FCCO300TA, Hampton, N.H.). No feeder layer was used.

On the second day of culture, the cells were observed with a Leica DM-IL (Leica Microsystems USA, Bannockburn, Ill.) or Nikon TS-100F (Nikon USA, Melville, N.Y.) microscope (40-400× total magnification with Hoffman modulation contrast and phase contrast optics) to ensure that they were healthy and continuing to grow.

On the third day, the cells were fed. To feed the cells, the original media (usually dark yellow, indicating active cellular metabolism) was removed and discarded with a glass pipette connected to vacuum. Care was taken not to aspirate the attached cells. Next, 5 mL of new fresh complete media was added to the cells and then the flask was placed back in the incubator.

On the fourth day, the cells were generally split in a ratio of 1:10, with nine (9) parts being frozen for future use, and one (1) part being propagated in culture. To split the cells, the media from the flask was removed. Then, 1000 μL of trypsin (GIBCO #25300-062, Carlsbad, Calif.) was added to the T-25 flask in order to detach the cells attached to the bottom of the flask. The flask was then incubated at 37° C. 5% CO₂ for a total of 5 minutes. Next, 900 μL of trypsin and cells was transferred out of the flask into a 15 mL tube. To this tube, 9.1 mL of freezing media (95% complete media, 5% dimethyl sulfoxide (DMSO) (Sigma-Aldrich #D8418-100ML, St. Louis Mo.)) was added, inactivating the trypsin and bringing the total volume to 10 mL. Pipetting the cells up and down in each tube was used to break any cell clumps apart. The 10 mL of freezing media/cells was aliquoted into 1 mL volumes in ten (10) cryotubes and these were placed in a −80° C. freezer overnight. The cryotubes were then transferred to a −180° C. liquid nitrogen tank the following day. To the 100 μL of trypsin and cells remaining in the T-25 flask, 4.9 mL of fresh complete media was added, inactivating the trypsin and bringing the total volume back to 5 mL. The flask was then re-incubated at 37° C. and 5% CO₂.

### Example 4

### Chemical and Electrical Stimulation and Synchronization

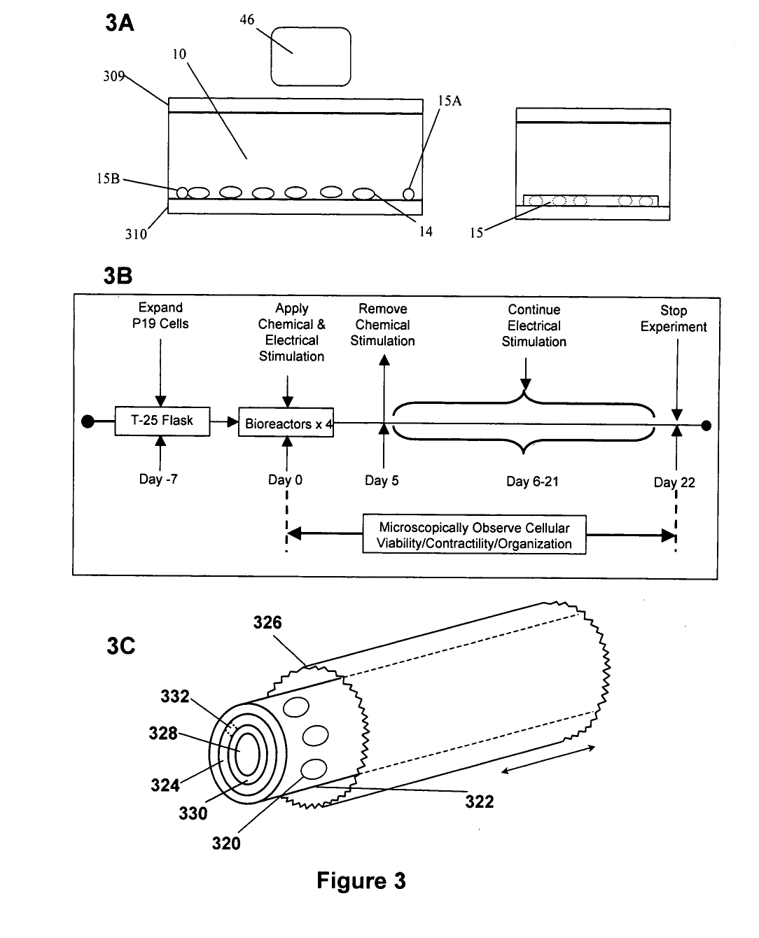

The reactor chamber used for this example is shown in FIG. 3A. As in FIG. 1, cells 14 are disposed in chamber 10 defined by a top 309 and bottom 310 for containing media and cell culture material. The cells are cultured within the electrical field created by electrical pulses to electrodes, shown in side view as 15A and 15B (anode and cathode), and in end view as 15. Microscope 46 provides optical data as to the process. Our experimental design for chemical and electrical stimulation is shown in FIG. 3B. On Day −7, P19 cells were thawed, grown, and split as outlined above. On Day 0, the P19 cells were washed three (3) times with phosphate buffered solution (PBS, pH 7.4) and then were transferred from complete media to differentiation media containing 1% DMSO. This media, known to differentiate cells into myocytes, was used to chemically stimulate the P19 cells for five (5) days.

Additionally, for the next 22 days, we continuously applied electrical pulses of varying field strengths (0-3 V/cm), widths (2-40 ms), and frequencies (10-25 Hz). The specific electric stimulation parameters are listed in Table 2.

TABLE 2
Electrical Stimulation Parameters.

| Electrical | | | | |
|---|---|---|---|---|
| Stimulation | Bioreactor | | | |
| Parameters | 1 | 2 | 3 | 4 |
| Pulse Width (ms) | 2 | 30 | 35 | 40 |
| Field Strength | 0, 1, 2, 3 | 0, 1, 2, 3 | 0, 1, 2, 3 | 0, 1, 2, 3 |
| (V/cm) | | | | |
| Pulse Frequency | 20 | 20 | 25 | 10 |
| (Hz) | | | | |

On Day 5, we exchanged the media containing DMSO with complete media (containing no DMSO) and continued the electrical stimulation. From Days 6-22, we visually assessed the cells for signs of viability, contractility, and organization. Spontaneously contracting P19-derived myocyte colonies were counted daily by one observer. We also documented our observations with the image acquisition system described above. Finally, we renewed either the differentiation media or complete media every three (3) days.

Electrical Synchronization

Electrical synchronization (pacing) was performed on Day 22 of culture on P19-derived myocytes and myocyte colonies in Bioreactor 1 only. This bioreactor was chosen because it demonstrated the most numbers of spontaneously contracting myocytes. These myocytes were also noted to be asynchronously contracting.

The electrical synchronization parameters are listed in Table 3.

TABLE 3
Electrical Synchronization (Pacing) Parameters
Electrical Synchronization Parameters

| Pulse Width | Field Strength | Pulse Freq | Capture? |
|---|---|---|---|
| (ms) | (V/cm) | (Hz) | (Y/N) |
| 2, | 0, 2.5, 5 | 2 | see Table 4 |
| 10-100 | 7.5, 10 | | |

A single channel electric cell pulser, identical in design to the four-channel pulser described above, was used to deliver the synchronization signals. The four channel pulser was disconnected and the single channel pulser was connected to the flat ribbon computer wire connected to each pair of gold electrodes from a given well of the bioreactor.

These signals consisted of square wave pulses having widths of either 2 ms or 10 to 100 ms (given in increments of 10 ms). Pulse field strengths of 0 to 10 V/cm were applied in increments of 2.5 V. Pulse frequency was set at a constant 2 Hz (corresponding to 120 contractions per minute).

As the different pulse parameters were applied, the myocytes were visually monitored via microscopy and were assessed for synchronization capture. Capture was defined as coordinated contractions of all myocytes at the applied frequency of 2 Hz. At baseline, the myocyte contraction rate ranged from zero (corresponding to no visually detectable contractions) to a maximum of 1.3 Hz (corresponding to 80 contractions per minute).

Documentation of synchronization was accomplished with two hundred (200) frame movies obtained at 20 frames/sec with QCapture Pro 5.1 software (QImaging, Burnaby, BC, Canada). The frames were stored on a custom-made computer equipped with a 3.4 GHz Pentium 4 processor, 2 GB RAM, and a 300 GB hard drive for further analysis.

Analysis of synchronized contractions was performed as follows. Two distinct colonies of P19-derived myocytes were identified and captured in a 200-frame movie as described above. The movie was taken before, during, and after synchronized contractions. The movie was then deconvoluted into individual frames using National Instruments Vision Assistant 7.1 software (National Instruments, Austin, Tex.). Next, using the same software, the first frame of the movie was used to create an edge detection algorithm. The algorithm was created by drawing one line on each colony such that each line overlapped with two (2) edges of each colony. The displacement of the colony edges with respect to the overlapping lines could then be determined for each frame. The displacements corresponded to contractions that could be seen in the photographs (data not shown). The edge detection algorithm was applied to all the frames in an automated fashion and the resulting displacements were recorded in a Microsoft Excel file (Microsoft Corp, Redmond, Wash.) for further analysis.

Chemical, Mechanical and Electrical Stimulation

FIG. 3C shows a bioreactor embodiment designed for chemical, mechanical and electrical stimulation. In this embodiment, cells 320 are cultured on or in a compatible surface (basement membrane and/or feeder cells) which is tubular in design. The surface 322 on which the cells are grown is attached to an elastomeric tube 324, which is housed in a bioreactor housing 326, and extends through the housing in order to be mechanically attached to movable devices, e.g. solenoids, for axially stretching the tube and/or to a pump for mechanically inflating and deflating the tube. The housing 326 is analogous to the chamber top and bottom in FIG. 3A, and is part of a bioreactor as shown in FIG. 1. Since the cell culture surface 322, preferably a cell culture basement membrane matrix, is attached to a flexible tube 324, which is made of, e.g. silicone tubing, the cells will be mechanically stretched. The tubing stretches in response to mechanical stress, both in the direction of the illustrated arrow, and in a radial direction. The mechanical stress is provided by the pulsatile pumping of fluid through interior chamber 328, which stretches the tubing in a more radial direction, and/or by stretching the tubing axially through actuators attached to either end. The electrode for delivering electrical stimulation is disposed, as shown at 330, is provided by a gold member 330 underneath the tubing. The electrode may comprise a gold member may be of a variety of shapes, and for purposes of illustration is shown as a tube concentric with the elastomeric tube 324. Openings 332 are provided in the elastomeric tube 324 in order to provide contact between the cell culture surface and the electrode.

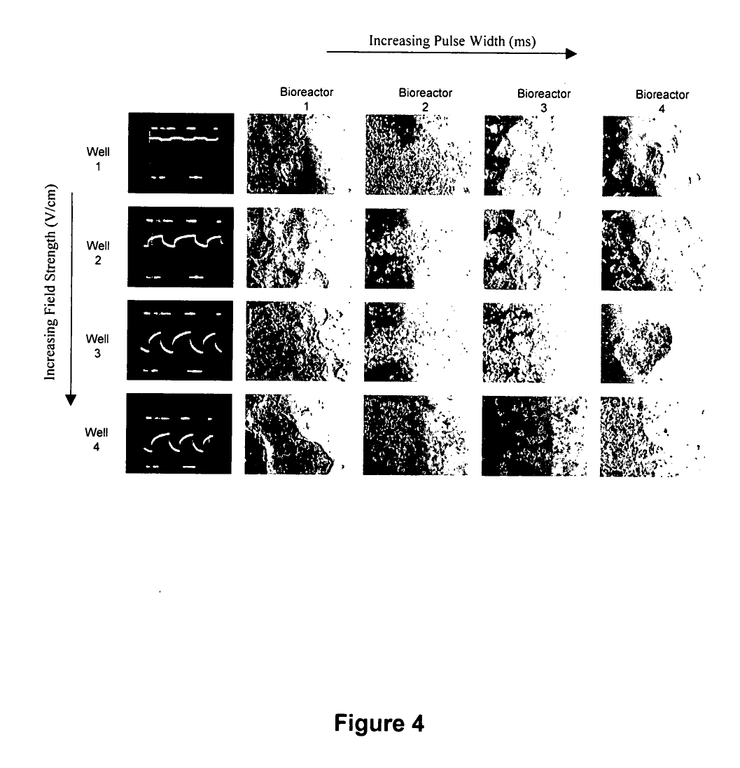

FIG. 4 shows a representative set of P19 progenitor cells exposed both to chemical and electrical stimulation. Over the course of the 22-day experiment, cell viability, as assessed by cell morphology, was inversely proportional to pulse width and field strength and had no apparent dependence on pulse frequency.

Thus, the experiments in Bioreactors 1-4 may be said to show that optimum electrical stimulation for the growth of P19-derived myocytes is a square pulse wave having a pulse width of 2 milliseconds or less and a pulse amplitude of 5 volts or less, and a frequency of 20 Hertz or less.

Bioreactor 1 was exposed to 1% DMSO for five (5) days and to electrical stimulation of pulse width 2 ms, field strengths of 0, 1, 2, and 3 V/cm, and pulse frequency of 20 Hz. Throughout the experiment, the cells in all the wells of this bioreactor were of uniform in size, attached to the bottom of the wells, and did not show any nuclear or cytoplasmic changes. Spontaneously contracting P19-derived myocyte colonies only appeared in Bioreactor 1 during the course of this experiment. Contracting myocytes could be detected in digital movie 1 (data not shown).

Bioreactor 2 was exposed to 1% DMSO for five (5) days and to electrical stimulation of pulse width 30 ms, field strengths of 0, 1, 2, and 3 V/cm, and pulse frequency also of 20 Hz. As the experiment progressed, the cells exposed to field strengths of 2 and 3 V/cm demonstrated nuclear condensation and cytoplasmic fragmentation and by Day 22, appeared non-viable. In addition, these same cells gradually lost their ability to adhere to the bottom of the wells. The cells exposed to 0 and 1 V/cm appeared healthy but did not exhibit any spontaneous contractions.

Bioreactor 3 was exposed to 1% DMSO for five (5) days and to electrical stimulation of pulse width 35 ms, field strengths of 0, 1, 2, and 3 V/cm, and pulse frequency of 25 Hz. As the experiment progressed, the cells exposed to field strengths of 1, 2 and 3 V/cm also demonstrated nuclear condensation, cytoplasmic fragmentation, and inability to attach. By Day 22, the cells exposed to 2 and 3 V/cm appeared non-viable and the cells suspension was dark; the cells exposed to 0 and 1 V/cm showed some healthy cells.

Bioreactor 4 was exposed to 1% DMSO for five (5) days and to electrical stimulation of pulse width 40 ms, field strengths of 0, 1, 2, and 3 V/cm, and pulse frequency of 10 Hz. Only two days into the experiment, the cells exposed to field strengths of 1, 2 and 3 V/cm demonstrated nuclear condensation, cytoplasmic fragmentation, and the inability to attach. By Day 22, all the cells except those exposed to 0 V/cm appeared non-viable as exemplified by extensive cellular fragmentation. In addition, by this time point, the media had turned a dark brown color, which was a marked departure from its usual pink color.

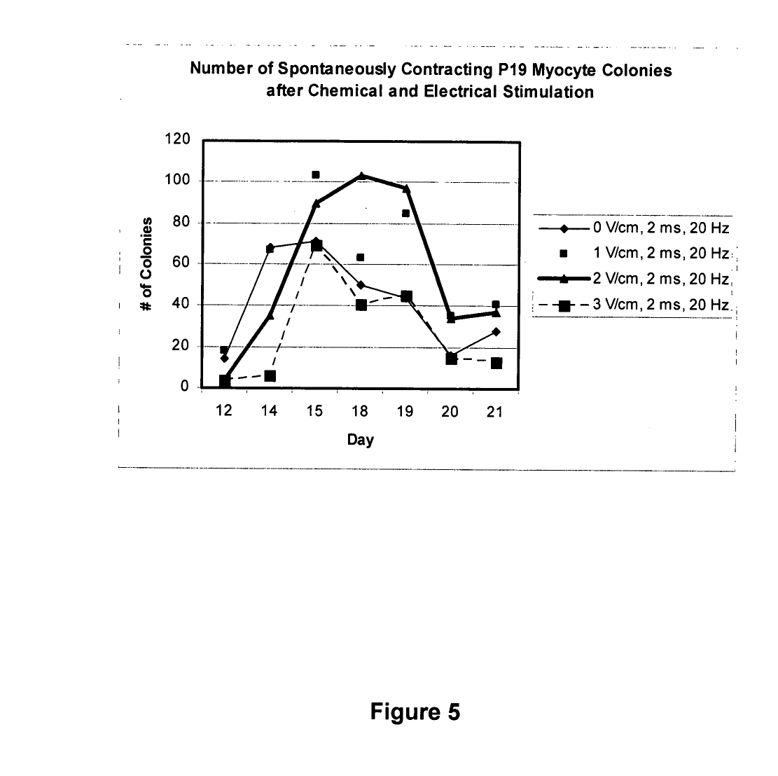

As shown in FIG. 5, spontaneously contracting P19-derived myocyte colonies appeared in Bioreactor 1 in all wells on Day 12. The number of colonies was greatest in the cells exposed to field strengths of 1 and 2 V/cm; these cells reached their maximum number on Days 15 and 18, respectively. Since the colonies were counted by only one observer, no statistical results could be reported.

Electrical Synchronization

Table 4 shows the electrical synchronization results. For pulse widths less than 40 ms, capture (i.e., tissue contraction in response to an electrical signal) could not be achieved at any field strength.

TABLE 4
Electrical Synchronization Results
Electrical Synchronization Results

| Pulse Width | Field Strength | Pulse Freq | Capture? |
|---|---|---|---|
| (ms) | (V/cm) | (Hz) | (Y/N) |
| 2, 10-40 | 0 | 2 | N |
| | 2.5 | | N |
| | 5 | | N |
| | 7.5 | | N |
| | 10 | | N |
| 50-100 | 0 | 2 | N |
| | 2.5 | | N |
| | 5 | | N |
| | 7.5 | | Y |
| | 10 | | Y |

Additionally, at field strengths of less than or equal to 5 V/cm, capture could also not be achieved with any pulse width.

The threshold for capture occurred for signals having field strengths of 7.5 and 10 V/cm, pulse widths 50-100, and frequency of 2 Hz. Cells uniformly exposed to these parameters could be synchronized. Synchronization was only performed for a few minutes; long-term synchronization was reserved for future experiments.

A movie made as described showed two P19-derived myocyte colonies that were synchronized; contractions are shown before, during, and after application of effective electrical synchronization as shown in Table 4 and discussed above. The correlation coefficient of contractions between the colonies before electrical synchronization was −0.6, indicating a non-statistically significant correlation in contractions. In contrast, the correlation coefficient of contractions between the colonies during synchronization was 0.6, indicating a statistically significant correlation in contractions and therefore, synchronization. Finally, the correlation coefficient of contractions between the colonies after synchronization was 0.5, also indicating a statistically significant correlation in contractions after being synchronized. This correlation was a positive by-product of prior synchronization.

### Example 5

### Bioreactor and Culture System with Pulsatile Flow

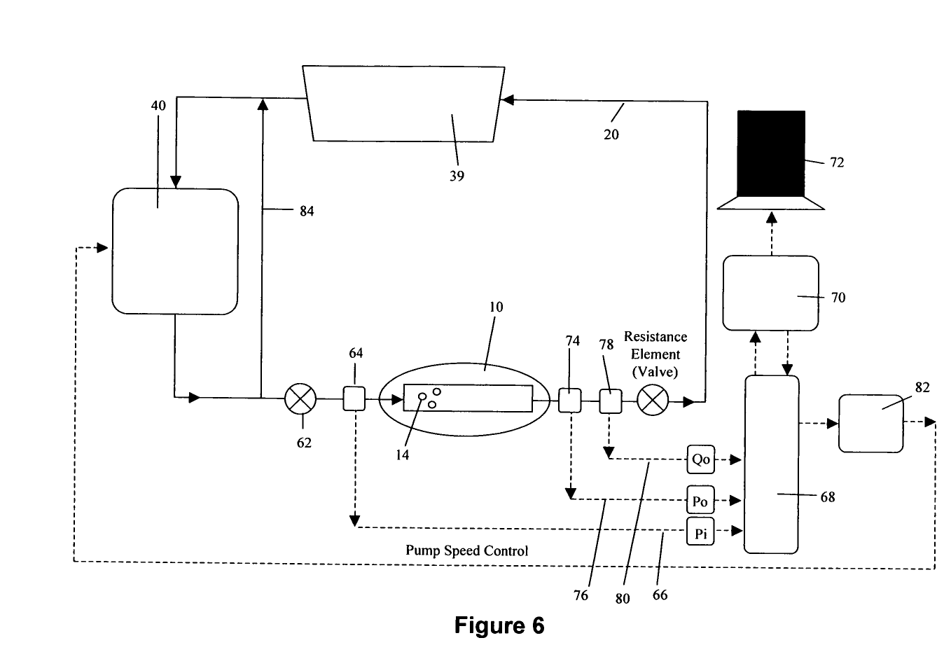

A bioreactor consisting of a pulsatile pump, tubing, inlet and outlet pressure transducers, an outlet flow probe, a data acquisition system, a microscope, and a high-speed digital charged-couple device (CCD) camera (for image acquisition and video microscopy was built). A schematic of the bioreactor layout is shown in FIG. 6.

Referring now to FIG. 6, a cell culture system is shown which illustrates various components as shown in FIG. 1, along with pressure modulating and monitoring devices. A culture chamber 10 containing, e.g. mESC cells 14, is connected to a media chamber, which also serves as a gas exchanger, 39. A pulsatile pump 40, again as shown in FIG. 1, is on the fluid flow circuit 20. Media or PBS can be pumped into the culture chamber 10. A valve 62 controls fluid flow from the pump to the culture chamber and acts as a resistance element. A pressure transducer at the culture chamber 10 inlet provides an inlet pressure reading on an electrical circuit 66 to a data acquisition device 68 connected to a microprocessor having, as is customary, a cpu and software for monitoring and controlling flow parameters. A computer display is connected to the cpu, as shown at 72. A outlet pressure transducer 74 provides an outlet pressure reading through circuit 76 to outlet pressure input to the data acquisition device 68. A flow meter 78 downstream of the outlet also provide a flow reading through circuit 80 to the data acquisition device 68. A pump controller 82 is electronically coupled to receive input from the data acquisition device 68 and to control the pump 40 to determine the timing and duration of pump pulses. The pump output may be wholly or partially directed through a shunt 84 to the media reservoir, bypassing the culture chamber 10.

The pulsatile pump used in this work was a Harvard Apparatus Model 1405 (Harvard Apparatus, Holliston, Mass.) modified for computer control with a Minarek MM10-115AC-PCM drive (Minarek Drives, South Beloit, Ill.) capable of generating physiologic pulsatility. The stroke volume ranged from 0.5-10.0 mL, the stroke rate could be varied from 20-200 cycles/min (cpm), and the flow rate could be adjusted from 10-2000 mL/min.

The tubing consisted of Tygon R3603 with an inner diameter ranging from ¼″ to ⅛″ and a wall thickness of 1/16″ (Cole-Parmer #EW-95903-06, #EW-06408-50, Vernon Hills, Ill.). The tubing was secured to each other and to the other bioreactor components via male and female barbed Luer locks (Cole-Parmer #EW-06359-35, #EW-30504-10, #EW-30505-76, Vernon Hills, Ill. and World Precision Instruments #14011, Sarasota, Fla.). The inlet and outlet pressure transducers were obtained from Abbott Labs Kit #42585-05. These transducers were capable of measuring the goal systolic pressures of 100-200 mmHg. The outlet flow probes and meter were a Transonic Ultrasonic Flow Probe (⅛″ outer diameter) and a Transonic T101 meter (Transonic Systems Inc, Ithaca, N.Y.). This probe and meter allowed measurement of flow rates of 0-400 mL/min.

A data acquisition system was used to monitor the pressure levels and flow rates. Our system consisted of National Instruments cFP-2000 control module hardware and National Instruments LabView 7.1 software (National Instruments, Austin, Tex.).

Finally, the bioreactor microscope was a Leica DM-IL (Leica Microsystems USA, Bannockburn, Ill.) inverted microscope fitted with 10× oculars, and 4×, 10×, 20×, and 40×objectives; this combination of optics allowed magnification of 40×, 100×, 200×, and 400×, respectively. Attached to the microscope was a Retiga 2000R high-speed digital CCD camera (QImaging, Burnaby, BC, Canada) capable of taking single frames and/or video-quality movies (30 frames/sec).

Three-Dimensional (3D) Culture System Assembly

To assemble the three-dimensional (3D) culture system, we first obtained individual off-the shelf items. We obtained a four-well Lab-Tek™ Chamber-Slide system (Nalge Nunc # 177437, Rochester, N.Y.). This chamber-slide system uses a chamber made of polypropylene and a slide made of Permanox™ (which reduces autofluorescence, a consideration for future experiments involving fluorescence detection of intracellular and extracellular makers). We then used a standard drill-press fitted with a ⅛″ drill bit to drill holes on each side chamber into the four wells (one (1) inlet and one (1) outlet per well, eight (8) total holes). Next we cut off the barbs of eight (8) female Luer lock fittings (Cole-Parmer #EW-06359-35, Vernon Hills, Ill.) and slid the modified fittings into the holes we created. Finally, we used Loctite™ RTV clear silicone adhesive and Loctite™ Hysol M-31CL clear medical epoxy (Loctite-Henkel, Rocky Hill, Conn.) to attach the fittings, the chamber, the chamber lid, and the slide all together to obtain the completed assembly.

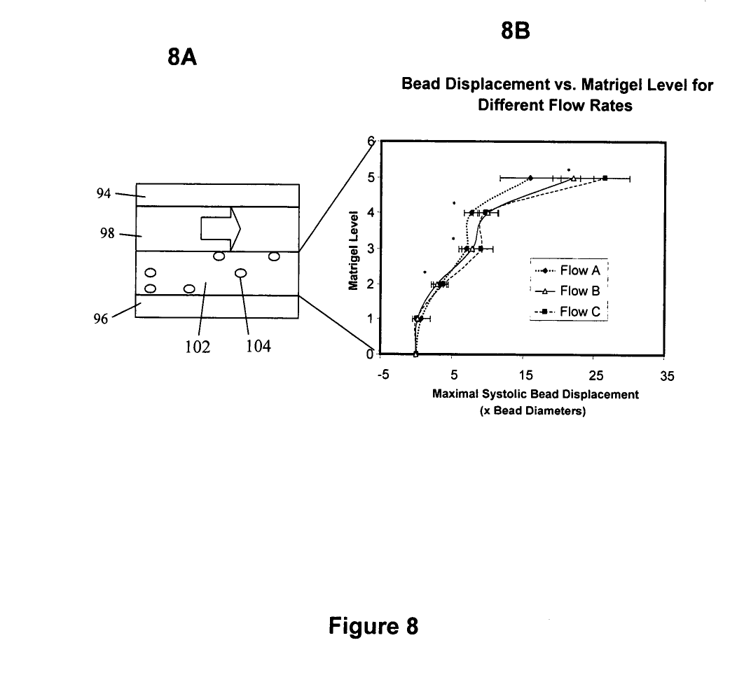

FIG. 7 shows another schematic of the set-up of a bioreactor 10 with the 3-D culture matrix. A Retiga 200R digital CCR camera 92 is positioned next to a culture chamber having a top 94 and a bottom 96, inlet 98 and outlet 100, defining a chamber holding a 3D cell culture matrix 102 at the bottom of the chamber which has embedded therein cells 104. The culture media 106 flows in the direction of arrow 108. The dimensions of a present embodiment are given, although alternative embodiments, including microfluidic channels and wells, may be created, given the present teachings. Further details are shown in FIG. 8. FIG. 8 shows, on the left, a schematic of the side view of the culture system. This schematic shows the beads dispersed at various levels within the Matrigel™ basement membrane culture matrix layer. It is important that the cells grow into a multilayer cluster, for later tissue engineering. Depth in the layer also affects cell movement. On the right side of FIG. 8, maximum bead displacement versus Matrigel™ basement membrane culture matrix level for flow rates of 30, 35, and 40 mL/min (Flows A, B, C, respectively) is shown. It is also understood that electrodes, as shown in FIG. 3, are also included in the structure. Referring now to FIG. 8A, bead displacement is plotted versus Matrigel™ basement membrane culture matrix level for the three flow rates of 30, 35, and 40 mL/min. Polymethylmethacrylate beads (6 μm diameter) were suspended randomly throughout a Matrigel™ basement membrane culture matrix layer of approximately 2.5 mm thickness in a chamber well. Pulsatile fluid flow was then applied along the top of the culture matrix layer, subjecting the layer to shear stress and horizontal displacement. The maximum displacements of the beads at various levels in the layer were recorded by video microscopy. At the culture matrix-chamber bottom interface (Level 0), the maximum displacement for each flow was essentially zero. At Level 5 near the culture matrix-fluid flow interface, the maximum displacement was largest (approx 20× bead diameters or 120 μm). The displacements at Levels 2-5 were statistically different than the displacements at Level 0 (*P<0.02). The differences in displacements between the three flow rates did not reach significance.

At the interface between the chamber bottom and the Matrigel™ basement membrane culture matrix layer (Level 0) of the culture system, the maximum bead displacement for each flow was essentially zero (0). At increasing basement membrane culture matrix levels (Levels 1-5), the maximum bead displacement within each level increased. At Level 5, near the interface between the culture matrix layer and fluid flow, the maximum displacement for each flow reached its largest value of approximately 20× bead diameters or 120 μm. This could be confirmed in the compiled AVI movies (data not shown).

The differences in displacements between Levels 0 and 1 were not statistically significant, however the displacements at Levels 2-5 were statistically different than the displacements at Level 0 (P<0.02). The differences in displacements between the three flow rates did not reach statistical significance.

### Example 6

### Complete Media

In order to perform cell culture in the pulsatile system, we prepared complete media as follows. The media consisted of Knock-Out Dulbecco's Minimal Essential Media (KO-DMEM) (Invitrogen #10829-018, Carlsbad, Calif.) supplemented with either 10% Fetal Bovine Serum (FBS) (GIBCO #26140-079, Carlsbad, Calif.) or 15% Serum Replacement (SR) (Invitrogen #10828-028, Carlsbad, Calif.). Next, to the above mixture, L-glutamine (GIBCO #25030-081, Carlsbad, Calif.) and non-essential amino acids (NEAA) (GIBCO #11140-050, Carlsbad, Calif.) were added (both were diluted from a 100× concentration of stock solution to a final concentration of 1× in the complete media). Next, a mixture of penicillin-streptomycin (PS) (GIBCO #15140-122, Carlsbad, Calif.) was added (diluted from a 100× concentration of stock solution to a final concentration of 1× in the complete media). Finally, to keep the stem cells undifferentiated in culture, 1000 U/mL of Leukemia Inhibitory Factor (LIF) (Chemicon #ESG1106, Temecula, Calif.) was added to the complete media.

### Example 7

### Pulsatile Cell Culture

In order to perform cell culture, we first obtained a 1 mL vial of frozen D3 mouse embryonic stem cells (mESC) from the American Type Culture Collection (ATCC # CRL-1934, Manassas, Va.). The vial of cells was thawed in a 37° C. water bath and the cells were then re-suspended in 9 mL of new complete media. Next, to culture the cells, 3 mL of 0.1% gelatin (Sigma-Aldrich #G1890-100G, St. Louis, Mo.) was placed into 2 wells of a 6-well plate (Becton Dickinson Biosciences #353224, Bedford, Mass.). The gelatin was kept in the wells for approximately 10 minutes and then aspirated. Then, 5 mL of the suspension of mESC was added into each of the 2 wells. The cells were then placed in a 37° C., 5% CO₂ incubator (Fisher Scientific Isotemp FCCO300TA, Hampton, N.H.) in order to promote their growth. No feeder layer was used.

On the second day of culture, the cells were observed with a Leica DM-IL (Leica Microsystems USA, Bannockburn, Ill.) or Nikon TS-100F (Nikon USA, Melville, N.Y.) microscope (40-400× total magnification with Hoffman modulation contrast and phase contrast optics) to ensure that they were healthy and continuing to grow.

On the third day, the cells were fed. To do so, the cells and original media (usually dark yellow, indicating active cellular metabolism) was removed and kept in a 15 mL tube (one for each well). Each tube was then spun down in a VWR Clinical 200 centrifuge (VWR #82013-812, West Chester, Pa.) at 300×g (corresponding to 1750 revolutions per minute (rpm) based on the size of the centrifuge rotor) for 3 minutes. The older media was then aspirated while the pellet of cells was left in the tube. Next, 5 mL of new fresh complete media was added to each tube. The clump of cells was then dissociated by pipetting up and down. The dissociated cells and new media were then transferred back into the original 2 wells of the 6-well plate.

On the fourth day, the cells were again observed via microscopy to ensure they were healthy and continuing to grow.

On the fifth day, the cells were generally split, with half being frozen for future use, and the other half being propagated in culture. To split the cells, the media and loose cells from each well were pipetted out and placed in their own 15 mL tube (for a total of two separate tubes). Then, 300 μL of trypsin (GIBCO #25300-062, Carlsbad, Calif.) was added to each well in order to detach any cells attached to the plate. After adding the trypsin, the wells were incubated at 37° C. 5% CO₂ for a total of 5 minutes. Then, after taking the wells out of the incubator, 2 mL of complete media was added to each well in order to inactivate the trypsin. After incubating the complete media with the cells and trypsin, the mixture was aspirated with a pipette and placed into its corresponding tube. If not all the cells were detached, 1 mL of pH 7.4 phosphate buffered solution (PBS) (GIBCO #10010-023, Carlsbad, Calif.) was used to further wash the cells and pipetting was used to detach them from the wells. The PBS and detached cells were then transferred into the well's corresponding tube. Then, each tube for each well was centrifuged at 300×g for 3 minutes. The supernatant of media and PBS was then suctioned from each tube, leaving the pellet of cells intact at the bottom of the tube. In one of the tubes, 10 mL of fresh, complete media was added, while in the other tube, 1 mL of freezing media (95% complete media, 5% dimethyl sulfoxide (DMSO) (Sigma-Aldrich #D8418-100ML, St. Louis Mo.) was added. Pipetting the cells up and down in each tube was used to break the cells apart. Then, 5 mL of new complete media containing the re-suspended cells was added to each of the two wells of the 6-well plate. The 1 mL of freezing media containing the other cells was transferred into a cryotube and placed into a −80° C. freezer overnight and then transferred to a −180° C. liquid nitrogen tank the following day. The 6-well plate containing the 2 wells of cells was re-incubated at 37° C. and 5% CO₂.

### Example 8

### Verification of Undifferentiated Cells

Markers of undifferentiated mESC consist of the presence of Alkaline Phosphatase (AP), Stage-Specific Embryonic Antigen-1 (SSEA-1), and Oct-4, and the absence of SSEA-3, SSEA-4, TRA-1-60, TRA-1-81. In order to verify that we had a population of undifferentiated mESC, we stained the cells with AP (Chemicon #SCR004, Temecula, Calif.). Undifferentiated cells stained with AP appeared red while the absence of staining indicated differentiated cells were present. For our experiments we used a population consisting mostly of undifferentiated mESC.

### Example 9

### Application of Flow to Beads in 3D Matrigel™ Basement Membrane Culture Matrix

For these experiments, we first diluted one (1) drop of non-fluorescent CaliBRITE polymethylmethacrylate beads having a diameter of six (6) μm (Becton Dickinson Biosciences, #340486, Bedford, Mass.) into one (1) mL of flow cytometry BD FACSFlow sheath fluid (Becton Dickinson Biosciences, # 342003, Bedford, Mass.) as per the manufacturers' instructions. Next, we suspended fifty (50) μL of the diluted bead solution randomly throughout one hundred-fifty (150) μL of liquid Matrigel™ basement membrane culture matrix (Becton Dickinson Biosciences # 354234, Bedford, Mass.). The suspension was performed in a 4° C. refrigerated cold room in order to keep the Matrigel™ basement membrane culture matrix in a liquid state.

After we suspended the beads in the culture matrix, we transferred the sample into one (1) well of the culture system assembly. This was also performed at 4° C. Immediately after transfer, the assembly was placed in a 37° C., 5% CO₂ incubator in order to allow the Matrigel™ basement membrane culture matrix to solidify. After 30 minutes, the solidification of the Matrigel™ basement membrane culture matrix was verified with the microscope at 200× and 400× magnifications. The thickness of the solidified Matrigel™ basement membrane culture matrix was approximately 2.5 mm. The beads could be seen randomly suspended in the Matrigel™ basement membrane culture matrix at various levels.

Next, an additional 250 μL of PBS was added to the well containing the bead suspension. After the culture system was prepared, it was connected to the bioreactor. The inlet of the well was attached to the outlet of the pulsatile pump and the outlet of the well was attached to the tubing returning fluid to the bioreactor reservoir. The culture system was then placed on the microscope stage and the well of interest was secured under the CCD camera.

To determine the short term effects (approximately 3 hrs) of pulsatile conditions on the bead suspensions, we then turned on the pulsatile pump while recording the effects with the CCD camera. Flow of PBS (approximately 250 mL in the media reservoir) was applied at 30, 35, and 40 mL/min, the pressure was set in the range of 120 mmHg systolic, and the rate of the pump was set at 50, 60, and 70 cycles per minute (cpm).

At six (6) levels (on average 400 μm apart) of the culture matrix, one-hundred fifty (150) frames were obtained at 30 frames/sec with QCapture Pro 5.1 software (QImaging, Burnaby, BC, Canada) in order to visualize the movement of the suspended beads. The frames were stored on a custom-made computer equipped with a 3.4 GHz Pentium 4 processor, 2 GB RAM, and a 300 GB hard drive for further analysis.

Each one-hundred-fifty (150) frame segment (corresponding to each layer in the culture matrix) was compiled into an AVI movie using Microsoft Windows Movie Maker 5.1 software (Microsoft Corp, Redmond, Wash.); the movies were visually inspected for maximum bead displacement and then individual frames were identified and analyzed for confirmation.

When the frames showing maximum displacement were identified, their displacement was measured with ImageJ imaging software (Rasband, W. S., U.S. National Institutes of Health, Bethesda, Md., USA, http://rsb.info.nih.gov/ij/, 1997-2005). Maximum displacement along the flow axis was calculated during systole and plotted at various levels through the matrix and normalized to the bead diameter for uniformity.

### Example 10

### Application of Flow to Cells in 3D Matrigel™ Basement Membrane Culture Matrix

For these experiments, we cultured mESC into embryoid bodies (clusters of mESC). Next we obtained approximately 200,000 mESC and suspended half of them in 250 μL of liquid Matrigel™ basement membrane culture matrix (Becton Dickinson Biosciences # 354234, Bedford, Mass.). The suspension was performed in a 4° C. refrigerated cold room in order to keep the Matrigel™ basement membrane culture in a liquid state. The other 100,000 mESC were suspended in complete media alone.

After suspending the cells in the Matrigel™ basement membrane culture matrix and complete media, we transferred each sample into one (1) well each of the culture system (2 wells total). This was also performed at 4° C. Immediately after transfer, the culture system was placed in a 37° C., 5% CO₂ incubator in order to allow the Matrigel™ basement membrane culture to solidify. After 30 minutes, the solidification of the Matrigel™ basement membrane culture was verified with the microscope at 100× magnification. The mESC could be seen suspended in the Matrigel™ basement membrane culture in one well and freely moving around in the well containing only mESC and media (not shown). Next, an additional 250 μL of media was added to each well.

After the wells were prepared, the culture system was connected to the bioreactor. We attached the inlets of the wells to the outlet of the pulsatile pump and attached the outlets of the wells to the tubing returning fluid to the bioreactor reservoir. The culture system was then placed on the microscope stage and the wells of interest were secured under the CCD camera.

To determine the short-term effects (approximately 3 hrs) of pulsatile flow and pressure on the mESC suspensions, we then turned on the pulsatile pump while recording the effects with the CCD camera. The flow of complete media (approximately 250 mL in the bioreactor reservoir) was controlled in the range from approximately 0-30 mL/min and the pressure was set in the range of 100-200 mmHg systolic. The rate of the pump was set at 60 cpm.

By placing the mESC in a 3D Matrigel™ basement membrane culture matrix or media alone, we were able visualize the response of the cells to applied physiologic pulsatile pressure and flow. As soon as pulsatile flow was applied to the cells suspended only in complete media, the cells washed away. Approximately 50% of the cells washed away immediately by visual inspection of the entire movie. In addition, the cells dispersed in media alone and did not form or re-form embryoid bodies (clusters of mESC).

In contrast to the above description, the cells in the Matrigel™ basement membrane culture matrix were constrained to the culture system well, moved in unison with the flow, and were not washed downstream. Also, the cells dispersed in the Matrigel™ basement membrane culture matrix formed embryoid bodies, a step which is important in differentiation of stem cells.

Approximation of Maximum Wall Shear Stress (Pulsatile)

Table 5 shows the estimated WSS calculated from the three flow rates (30, 35, 40 mL/min) and the culture system well geometry (well height=7.5 mm, well width=7.0 mm) used in this study (A-C) and the estimated WSS calculated using different flow rates (50, 75, 100 mL/min) and well geometries (well height=2.0 mm, well width=7.0 mm) (D-F).

TABLE 5

| | Present Study | | | Future Studies | | |
|---|---|---|---|---|---|---|
| | A | B | C | D | E | F |
| Flow Rate (mL/min) | 30 | 35 | 40 | 50 | 75 | 100 |
| Well Height (mm) | 7.5 | 7.5 | 7.5 | 7.5 | 2.0 | 2.0 |
| Well Width (mm) | 7.0 | 7.0 | 7.0 | 7.0 | 7.0 | 7.0 |
| WSS with Water | 0.07 | 0.09 | 0.1 | 0.1 | 2.7 | 3.6 |
| Viscosity (dyne/cm²) | | | | | | |
| WSS with Blood | 0.3 | 0.4 | 0.4 | 0.5 | 10.7 | 14.3 |
| Viscosity (dyne/cm²) | | | | | | |

In order to approach mouse aortic WSS (5-25 dynes/cm²), higher flow rates, a smaller well height, and a higher viscosity will be needed in future studies. Higher flow rates can easily be achieved by increasing the pulsatile pump rate and/or stroke volume. The effective well height can be readily achieved by making the overall chamber height shorter and/or adding more Matrigel™ basement membrane culture to the well (in order to make the cross-section of the well flow path shorter). The viscosity can be increased by adding Dextran to the circulating culture media to make a 5% solution, which approximates the viscosity of blood.

Summary of Reagents

TABLE 6

| Description | Clone | Vendor |
|---|---|---|
| Mouse Embryonic Stem Cells | | |
| ES-D3, non germ-line competent, | D3 | ATCC |
| original deposit from Doetschman | | |
| ES-D3, germ-line competent, | D3 | ATCC |
| derived from CRL-1934 | | |
| ES-D3, deposited by Chambon | D3 | ATCC |
| Mouse Embryonic Fibroblasts | | |
| CF-1 mouse embryonic fibroblasts | MEF | ATCC |
| | (CF-1) | |
| Embryonic fibroblast-derived | STO | ATCC |
| cell line | | |
| Cell Culture Media | | |
| KO-DMEM, 500 mL | — | Biostores/Invitrogen |
| KO-Serum Replacement, 500 mL | — | Biostores/Invitrogen |
| FBS, US Qualified, 500 mL | — | Biostores/GIBCO |
| 2-Mercaptoethanol, 50 mL | — | Biostores/Invitrogen |
| L-Glutamine 200 mM, 100 mL | — | Biostores/GIBCO |
| MEM Non-essential amino acids, | — | Biostores/GIBCO |
| 100×, 100 mL | | |
| Pen/strep, 100 mL | — | Biostores/GIBCO |
| Sodium Bicarb Solution, 7.5% | — | Biostores/GIBCO |
| Alpha-MEM, 500 mL | — | Biostores/GIBCO |
| PBS pH 7.4, 1×, 500 mL | — | Biostores/GIBCO |
| PBS pH 7.4, 10× | — | Biostores/GIBCO |
| Dimethyl sulfoxide | — | Biostores/ |
| | | Sigma-Aldrich |
| Undifferentiated | | |
| mESC Growth Factors | | |
| LIF (ESGRO) | — | VWR/Chemicon |
| EC Progenitor | | |
| Growth Factors | | |
| VEGF | — | VWR/Chemicon |
| SMC Growth Factors | | |
| PDGF-BB | — | VWR/Chemicon |
| dibutyryl-cyclicAMP (db-cAMP), | — | Biostores/ |
| 0.5 mM | | Sigma-Aldrich |
| Retinoic acid (Vitamin A), | — | Biostores/ |
| 10 nM? 0.5 microM? | | Sigma-Aldrich |

### Example 11

### From Cell Culture to Blood Vessel

FIG. 9 shows a diagrammatic representation of an application of the present cell culture and selection system in the preparation of an assembled blood vessel. As can be seen, a cell culture system 120 as exemplified in FIG. 7 is seeded with stem cells and differentiated into endothelial cells, smooth muscle cells, or fibroblasts, in different reactors or different runs in the same reactor. The reactor is equipped with electrodes for providing an electrical field across the cell culture and the cells are cultured under pulsatile flow as described above. A fibroblast layer, an electrically responsive smooth muscle layer, and an endothelial layer are prepared in culture. As shown at 122 the layers are removed from cell culture individually, and combined in a tube to provide assembled layers and an assembled vessel, containing an adventitia, media and intima layer. When the appropriate layer has been cleared of undifferentiated cells by the present selection methods, the individual cell layers are harvested. They are then assembled into anatomically correct layers to form an assembled vessel having intima, media and adventitia layers.

In another embodiment, described in Example 13 below, a cardiac muscle graft is prepared in tubular form as well, but only need comprise a single layer of cardiac myocyte tissue cultured in three dimensional matrix under the conditions described above (mechanical and electrical stimulation) until tissue contraction is observed.

### Example 12

### Culture of Human Stem Cells into Cardiac Myocytes

In this example, human stem cells are cultured in the present system and separated magnetically to yield a population of cardiac myocytes derived from the stem cells. The human stem cells are preferably patient specific, and the derived myocytes are implanted into the patient in order to help repair damaged heart tissue. The stem cells are obtained from a side population (SP) of human bone marrow cells, as described in Jackson et al., “Regeneration of ischemic cardiac muscle and vascular endothelium by adult stem cells,” J Clin Invest, June 2001, Volume 107, Number 11, 1395-1402. Side-population (SP) cells are selected based on the rapid efflux of the fluorescent DNA-binding dye Hoechst 33342. The engrafted SP cells (CD34−/low, c-Kit⁺, Sca-1+) or their progeny migrated into ischemic cardiac muscle and blood vessels, differentiated to cardiomyocytes and endothelial cells, and contributed to the formation of functional tissue in mice. Therefore, these cells are expected to differentiate into cardiac myocytes under the proper culture and separation procedures, as described above.

Alternatively, human embryonic stem cells (hESC) may be cultured in the present system and separated magnetically. A protocol for the culture of HESC into cardiomyocytes is described in Xu et al., “Characterization and Enrichment of Cardiomyocytes Derived From Human Embryonic Stem Cells”, (Circulation Research, 2002; 91:501-508) and Kofidis et al., “Allopurinol/uricase and ibuprofen enhance engraftment of cardiomyocyte-enriched human embryonic stem cells and improve cardiac function following myocardial injury”, (Eur J of Cardio-Thoracic Surgery, 2006; 29:50-55). In these publications, HESC are formed into embryoid bodies (EB) via suspension in low attachment plates for 4 days in a prescribed culture medium. After 4 days in suspension, EBs are transferred onto gelatin or poly-L-lysine-coated plates and then differentiated for less than a week with either dimethyl sulfoxide (DMSO), all-trans retinoic acid (RA) or 5-aza-2′-deoxycytidine (5-aza-dC. Next, the differentiation factor (DMSO, RA or 5-aza-dC) is removed and then the cells are monitored for the presence of beating cells. The resulting cardiomyocytes are further enriched via separation from non-differentiated hESC in a discontinuous Percoll gradient. Muscle markers are evaluated using dissociated hES cell-derived cardiomyocytes: cardiac-specific troponin I (cTnI) myosin heavy chain (MHC), tropomyosin, α-actinin, desmin, connexin-43, and cardiac troponin T (cTnT) proteins are detected in single beating cells or clusters of cells. hES cell-derived cardiomyocytes also specifically express several cardiac transcription factors, including GATA-4, MEF-2, and Nkx2.5, in the differentiated cultures. Injection of the hESC-derived cardiomyocytes into ischemic rodent myocardium contributes to the formation of functional tissue. As with the SP population of bone marrow cells, the hESC are expected to differentiate into cardiac myocytes under the proper culture and separation procedures, as described above.

In summary, the undifferentiated stem cells are cultured in the bioreactor, and allowed to form cardiac myocytes by removing factors, which prevent differentiation, e.g. beta-FGF. As differentiation progresses, EBs will begin to dissociate from the adherent cells and become non-adherent. These are separated, preferably by magnetic labeling. The EBs prepared according to the present method will form clusters of beating cells. These are re-attached, cultured and mixed with appropriate materials to be cast into tissue grafts. The annular shape allows additional mechanical stimulus.

Alternatively, human CD133+ cells may be isolated via magnetic-activated cell sorting, AC133 Cell Isolation Kit (Miltenyi Biotech, Bergisch-Gladbach, Germany, http://www.miltenyibiotec.com), according to manufacturer's recommendations. A protocol for the culture of stem cells into cardiomyocytes is described in Shmelkov et al., “Cytokine Preconditioning Promotes Codifferentiation of Human Fetal Liver CD133+ Stem Cells Into Angiomyogenic Tissue,” (Circulation, 2005; 111:1175-1183.) This publication discloses human fetal liver CD133+ and CD133− cell subpopulations cultured with 5′-azacytidine or vascular endothelial growth factor (VEGF165) and/or brain-derived nerve growth factor (BDNF). CD133+ but not CD133− cells from human fetal liver codifferentiated into spindle-shaped cells, as well as flat adherent multinucleated cells capable of spontaneous contractions in culture. The resulting spindle-shaped cells were confirmed to be endothelial cells by immunohistochemistry analysis for von Willebrand factor and by acetylated LDL uptake. Multinucleated cells were characterized as striated muscles by electron microscopy and immunohistochemistry analysis for myosin heavy chain (MHC). Presence of VEGF165 and BDNF significantly enhanced angiomyogenesis in vitro. Inoculation of cells derived from CD133+ cells, but not CD133⁻ cells, into the ear pinna of NOD/SCID mice resulted in the formation of cardiomyocytes, as identified by immunostaining with cardiac troponin-T antibody. These cells generated electrical action potentials, detectable by ECG tracing.

Therefore, either isolated SP cells, hESC, or CD 133+ cells are cultured in the present bioreactor in the presence of DMSO, RA, 5′-azacytidine, VEGF165, and/or BDNF to produce cells which are either committed to or fully differentiated as cardiac myocytes. The cells in culture are subjected to an electrical pulse frequency of 20 Hz with a 2 ms pulse width and a field strength of 1V/cm. After five days of culture, the DMSO, RA, 5′-azacytidine, VEGF165, and/or BDNF are no longer added. The cells are cultured in a bioreactor comprising fibronectin-coated substrates adjacent the electrode, and subjected to pulsatile conditions by pump 36, at a low wall shear stress, which is increased over the culture period, which is expected to be approximately 18 days. The cells are exposed to antibodies to common markers of cardiac muscle (cardiac-specific troponin I (cTnI) myosin heavy chain (MHC), tropomyosin, α-actinin, desmin, connexin-43, cardiac troponin T (cTnT), GATA-4, MEF-2, and Nkx2.5), which have been marked with magnetic beads. The cells are then subjected to mild trypsinization (as described in Example 3) and circulated to a magnetic separator, where marker+ cardiac myocytes are removed, washed and resuspended in sterile buffer for infusion into the patient.

### Example 13

### Culture of Human Stem Cells into Cardiomyocytes and Tubular Cardiovascular Tissue

This example demonstrates the use of embryonic stem cells which are differentiated into cardiomyocytes (ESC-CMs), various basement membrane materials (e.g. Matrigel, Type I collagen), electrical stimulation and mechanical stimulation using a flexible cell substrate, as shown in FIG. 3C.

First hESC-CMs are prepared and optimized by mechanical stretch and electrical stimulation (Step 1). Undifferentiated stem cells are grown on a basement membrane, e.g. Matrigel™, Types I and IV collagen, and exposed to appropriate growth factors, e.g. vascular endothelial growth factor (VEGF), and subjected to mechanical and electrical stimulation. The resulting electrically responsive cells are used to engineer a 3D contractile tissue graft. Type IV collagen is added in order to enhance hESC-CM attachment and force transmission. VEGF is included in the culture media with the rationale that this growth factor will induce vascularization of the implanted graft. Finally, the present method uses electromechanical stimulation at the tissue level in order to improve graft survival and function.

It is thought that the addition of in vitro electromechanical stimulation that simulates the in vivo environment may improve hESC-CM yield and function by activating stretch ion channels, upregulating voltage-gated ion channels, and driving enhanced polymerization of cytoskeletal structures.

Human embryonic stem cells (hESCs) may be used for initial culture. For example, one may use a federally approved line (WA09, 46XX from Wicell), or a non-federally approved line, depending on the circumstances. The hESCs are preferably cultured initially on irradiated MEF feeder layers. For maintenance using feeder-free conditions, hESCs are then cultured as described in the literature, e.g. Xu et al., “Characterization and enrichment of cardiomyocytes derived from human embryonic stem cells,” Circ Res., Sep. 20, 2002; 91(6):501-508. For differentiation, hESC embryoid bodies (EB) are dispersed into cell aggregates and spontaneously contracting hESC-CMs will be identified as clusters in outgrowths of EBs starting at day 7. For enrichment of cardiomyocytes, EBs will be separated on a Percoll density gradient or using magnetic separation as described above.

Further teaching on the formation of EBs is found in U.S. Pat. No. 6,602,711 to Thomson, et al., issued Aug. 5, 2003, entitled “Method of making embryoid bodies from primate embryonic stem cells.”

U.S. Pat. No. 5,928,943 to Franz, et al., issued Jul. 27, 1999, entitled “Embryonal cardiac muscle cells, their preparation and their use,” discloses an alternative hanging drop method for forming embryoid bodies, and also describes the use of engineered hESCs, which may be employed in the alternative in the present method. The engineered hESCs described there contain two gene constructs comprising: a) a regulatory, 1.2-kb long DNA sequence of the ventricle-specific myosin light-chain-2 (MLC-2v) promoter, the selectable marker gene β-galactosidase in fusion with the reporter gene neomycin; and b) a regulatory DNA sequence of the herpes simplex virus thymidine kinase promoter and the selectable marker gene hygromycin.

Using standard techniques, hESCs may be made to express reporter genes for subsequent in vivo tracking by molecular imaging methods (Cao, F. et al., In vivo molecular imaging of human embryonic stem cell derived cardiomyocytes after transplantation into the ischemic myocardium. 57:1B-2B, 2006). These markers are used to track the cells in vivo and make them more tractable to imaging methods such as bioluminescence and microPET. hESCs can be differentiated into beating EBs in the presence of Noggin (500 ng/ml) and bFGF (40 mg/ml) (Yao, et al., “Long-term self-renewal and directed differentiation of human embryonic stem cells in chemically defined conditions,” Proc Natl Acad Sci USA, May 2, 2006; 103(18):6907-6912) and further enriched for beating CMs (˜45% pure) by Percoll separation. Expression of reporter genes does not affect ES cell viability, proliferation, or differentiation of hESCs into different germ layers after repeated passages (>50).

The isolated EBs are placed adjacent electrodes, if they have not been isolated within the present bioreactor, which contains electrodes. The preferred means for separation uses magnetic labeling in situ, as described above, but any suitable method may be used. The bioreactor, as discussed above, is controlled as to electrical stimulation and mechanical stretch, and preferably includes a feedback system that couples the timing between the inputs of electrical stimulation and mechanical stretch. The stretchable cell substrate is included in the bioreactor described above, which provides control over temperature, gas, and media delivery. Electrical sensing of hESC-CMs will be accomplished by the electrodes as shown in FIG. 3. Mechanical stretching of the cells and the contractile response of the cells are detected by microscope 46 (FIG. 1).

Voltage sensitive dyes may be used to confirm electrical parameters. Optimal conditions of mechanical stress, electrical pulse and growth factor addition may be determined by experimentation. For example, the function of conditioned hESC-CMs (CCMs) may be compared to unconditioned hESC-CMs (UCMs) (control). Initial electrical and mechanical inputs are listed in Table 7.

TABLE 7

| | Amplitude | Width | Freq. | Flow | Pressure | Shear stress | |
|---|---|---|---|---|---|---|---|
| Range of conditions | (V/cm) | (ms) | (Hz) | (type) | (mean, mmHg) | (dyne/cm2) | Strain (%) |
| Unconditioned | 0 | 0 | 0 | none | 0 | 0 | 0 |
| purified hESC-CM | | | | | | | |
| Conditioned purified | 1, 5, 10 | 2 | 1 | pulsatile | 80 | 10 | 3, 6, 9 |
| hESC-CM | | | | | | | |

Electrical outputs will be cardiomyocyte-generated action potentials. Mechanical outputs will be contraction rate and amplitude of beating cells. During optimization, cell morphology is assessed by immunostaining for cardiomyocyte specific markers such as troponin, MEF2c, α-actin, and connexin.

The pulsatile flow provided by the pulsatile pump to the inner annulus of the tubing stretches the tubing radially in a pulsatile fashion. Also, depending on the elasticity of the tubing, maximal stretching (strains) of the tubing can range from 1.5-15% at a given pressure of 150 mmHg and flow of approx 30 mL/min

hESC-CMs (optimized from Step 1) are then combined to engineer a 3D contractile tissue graft similar to what has previously been described by others (e.g. Zimmermann et al., “Tissue engineering of a differentiated cardiac muscle construct,” Circ Res., Feb. 8, 2002 2002; 90(2):223-230., Zimmermann et al., “Engineered heart tissue grafts improve systolic and diastolic function in infarcted rat hearts,” Nat. Med., 2006; 12(4):452; Guo et al., “Creation of engineered cardiac tissue in vitro from mouse embryonic stem cells,” Circulation, May 9, 2006; 113(18):2229-2237).

Unlike other methods, this step comprises the addition of (1) collagen IV to enhance hESC-CM attachment and force transmission; (2) vascular endothelial growth factor (VEGF) with the rationale that this growth factor will induce vascularization of the implanted graft; and (3) electrical stimulation for the purpose of temporally synchronizing the tissue grafts.

Collagen IV is commercially available. Collagen IV is a major constituent of the basement membranes along with laminins and enactins. It is composed of alpha 1 IV chain and alpha 2 IVchain in 2:1 ratio. It can form insoluble fibers with high tensile strength.

For specific guidance on the preparation and use of human recombinant VEGF, see Houck et al., “The vascular endothelial growth factor family: identification of a fourth molecular species and characterization of alternative splicing of RNA,” Mol. Endocrinol., 1991 December; 5 (12):1806-14.

hESC derived cardiomyocytes, embedded in a basement membrane mixture of collagen I, and matrigel (and/or collagen IV and VEGF) may be formed into a structure suitable for grafting into a blood vessel. This may be termed a “tissue graft” having a 3D nature made possible by the extracellular matrix components (collagen I, IV, matrigel). Just growing hESC on the tube alone would likely only give a monolayer of cells around the tubing. The cells and basement membrane and growth factor constituents may be prepared by casting in cylindrical molds which loosely contain silicone tubing which has been rendered self supporting with a relatively rigid solid insert (e.g. Teflon). Thus, the tissue, which has been prepared in step 1, is attached to the silicone tubing.

The cast cell mixture, which is formed into an annular shape, of a gel like material comprising the cultured hESC derived cardiomyocytes (CM), the cell substrate (containing collagen IV), undergoes further stimulation in a pulsatile flow system. The use of different sizes of silicone tubing will allow the eventual formation of two different sized 3D tissue grafts. In this example, 2.5×10⁶ cells comprise a tissue graft having a volume of approximately 0.9 mL.

After allowing the constituents to form over seven days in static culture (and no electrical stimulation), the silicone tubing is placed into apparatus for mechanical and electrical stimulation. At this point, the silicone tubing-cylindrical tissue will be connected in line to a pulsatile flow system (FIG. 3), where it will undergo 10% stretch and pulsed electrical stimulation (optimized from Step 1) for an additional seven days. Media is also circulated around the tissue grafts to provide additional nourishment. At the end of seven days of dynamic culture, the tissue grafts may either be left in their tubular form, to be implanted in the aortic position, or cut along their longitudinal axis to create an implantable cardiac tissue graft. The pulsatile flow system used to further culture the cells for the graft can deliver pulsatile flow rates in the ranges found in the developing human heart tube as well as found in adult cardiovascular tissues (where heart rate can vary from ˜250 bpm during development down to ˜60 bpm during adulthood). The system of FIG. 3 has been designed to deliver pulsatile flow to a specially designed chamber that allows intra- and extra-luminal flow of media to cardiovascular tissues attached to a hollow tube. In addition, the system has been designed to have accurate computerized control of pH, temperature, gas, and nutrient delivery. Electronics (FIG. 2) apply various waveforms (square, sinusoidal, dichrotic) at rates up to 240 cycles per minute and the device uses video microscopy to resolve differences in vessel wall stretch and strain over various rates.

The pulsatile flow system, similarly configured to systems used for tissue engineered blood vessels, is used to stretch cells radially and axially; the radial pulsatile stimulation allows scaling of mechanical stretch and will be more representative of the oriented loading found in vivo than the biaxial or uniaxial stretch systems previously described. The sizes of the silicone tubing are scalable and may be adjusted to match the required area of the cardiac tissue graft. Overall, the combined width, depth, and height of the tissue graft will guide the number of cells (˜2-5×10⁶ hESC-CM/graft) and amount of Matrigel, collagen, VEGF (I 0 μg/mL) that will ultimately be used in a given application.

In assessing the tissue graft, cardiomyocyte identity, confluence, and morphology is assessed by immunohistochemistry with cardiac specific markers as described above. Cell viability may be assessed by Annexin V-propidium iodide (PI) staining. Cellular ultrastructure and extracellular matrix morphology may be assessed by SEM and TEM as similarly described in Zimmerman et al., references cited above. VEGF may be detected by immunohistochemistry with commercially available antibodies.

Furthermore, the enriched population of hESC-CMs shows appropriate expression of cardiomyocyte markers and appropriate organization, as shown in FIG. 4. These cells have subsequently been tested for their ability to survive in vivo and improve cardiac function as described next.

Aligned films of collagen I can be layered to form sheets and tubes (see FIG. 9). In this design, alignment between layers has been structured with the same axial-radial asymmetry observed in cardiac tissue. Initial studies have shown these sheets and tubes formed from collagen I alone support stem cell adhesion and growth. P19 stem cells proliferated and adhered readily to the collagen structures. However, these collagen scaffolds alone do not provide the required mechanical compliance for stretching/straining cardiomyocytes. Thus, one should combine basement membrane materials, preferably Matrigel,™ collagen I, and collagen IV, to achieve the desired mechanical properties and provide physical cues leading to spatial organization of cells. Biomaterial scaffolds with incorporated vascular endothelial growth factor (VEGF) may be used to promote angiogenesis and regeneration. Scaffolds with incorporated growth factors have been shown to induce differentiation of ES cells, provide anchorage for adherent cells, and induce angiogenesis. The ability to induce angiogenesis is an attractive feature for the engraftment of tissue-engineered grafts into native tissue since these grafts will require a blood supply to maintain viability.

### CONCLUSION

The present specific description is meant to exemplify and illustrate the invention and should in no way be seen as limiting the scope of the invention, which is defined by the literal and equivalent scope of the appended claims. Variations upon the specific embodiments exemplified are apparent to those skilled in the art, given the present teachings.

For example, bacteria and other microorganisms could be cultured and separated in the present system using the magnetic cell separator. Outer membrane proteins and LPS in many gram-negative bacteria present targets that allow for separation on the basis of serotype. Nerve, neuroendocrine or other electrically responsive cells may be cultured according to the present disclosure regarding defined electromechanical stimulation. Various types of adherent or liquid cell culture media could be used. Different types of electrodes and magnets could be used for separation or inducement of electrical properties of cells organized into muscle or nerve tissue. Permanent magnets could be physically moved or exposed/unexposed to create pulses or cell separation. In particular, it should be noted that the cell separation based on magnetic labeling may be carried out in an iterative manner as the same cells pass though the separation zone multiple times. This permits more natural culture conditions.

### REFERENCES

1. American Heart Association. Heart Disease and Stroke Statistics-2005 Update. 2002.
2. Langer R, Vacanti J P. Tissue engineering. Science. 1993; 260(5110):920-926.
3. Vacanti J P, Langer R. Tissue engineering: the design and fabrication of living replacement devices for surgical reconstruction and transplantation. Lancet. 1999; 354:SI32-SI34.
4. Weitz J I, Byrne J, Clagett G P, et al., Diagnosis and treatment of chronic arterial insufficiency of the lower extremities: a critical review. Circulation. Dec. 1, 1996; 94(11):3026-3049.
5. Quinones-Baldrich W J, Busuttil R W, Baker J D, et al., Is the preferential use of polytetrafluoroethylene grafts for femoropopliteal bypass justified? J Vasc Surg. September 1988; 8(3):219-228.
6. Pevec W C, Darling R C, L'Italien G J, et al., Femoropopliteal reconstruction with knitted, nonvelour Dacron versus expanded polytetrafluoroethylene. J Vasc Surg. July 1992; 16(1):60-65.
7. Hamada Y, Kawachi K, Yamamoto T, et al., Effect of coronary artery bypass grafting on native coronary artery stenosis. Comparison of internal thoracic artery and saphenous vein grafts. J Cardiovasc Surg (Torino). April 2001; 42(2):159-164.
8. Weinberg C B, Bell E. A blood-vessel model constructed from collagen and cultured vascular cells. Science. 1986; 231 (4736):397-400.
9. L'Heureux N, Germain L, Labbe R, et al., In vitro construction of a human blood vessel from cultured vascular cells: A morphologic study. Journal of Vascular Surgery. 1993/3/1993; 17(3):499-509.
10. Niklason L E, Gao J, Abbott W M, et al., Functional arteries grown in vitro. Science. 1999; 284(5413):489-493.
11. Huynh T, Abraham G, Murray J, et al., Remodeling of an acellular collagen graft into a physiologically responsive neovessel. Nat. Biotechnol. November 1999; 17(11):1083-1086.
12. Hoerstrup S P, Zund G, Sodian R, et al., Tissue engineering of small caliber vascular grafts. European Journal of Cardio-Thoracic Surgery. 2001; 20(1):164-169.
13. L'Heureux N, Paquet S, Labbe R, et al., A completely biological tissue-engineered human blood vessel. Faseb Journal. 1998; 12(1):47-56.
14. Nerem R M, Seliktar D. Vascular tissue engineering. Annual Review of Biomedical Engineering. 2001; 3:225-243.
15. Zandstra P W, Nagy A. Stem cell bioengineering. Annu Rev Biomed Eng. 2001; 3:275-305.
16. MacNeill B D, Pomerantseva I, Lowe H C, et al., Toward a new blood vessel. Vasc Med. August 2002; 7(3):241-246.
17. Rafii S, Lyden D. Therapeutic stem and progenitor cell transplantation for organ vascularization and regeneration. Nat. Med. June 2003; 9(6):702-712.
18. Matsumoto K, Yoshitomi H, Rossant J, et al., Liver organogenesis promoted by endothelial cells prior to vascular function. Science. Oct. 19, 2001; 294(5542):559-563.
19. Lammert E, Cleaver O, Melton D. Induction of pancreatic differentiation by signals from blood vessels. Science. Oct. 19, 2001; 294(5542):564-567.
20. Rezai N, Podor T J, McManus B M. Bone marrow cells in the repair and modulation of heart and blood vessels: emerging opportunities in native and engineered tissue and biomechanical materials. Artif Organs. February 2004; 28(2):142-151.
21. Merchant A M, Flake A W. Surgeons and stem cells: a pragmatic perspective on shifting paradigms. Surgery. November 2004; 136(5):975-980.
22. Kannan R Y, Salacinski H J, Sales K, et al., The roles of tissue engineering and vascularisation in the development of micro-vascular networks: a review. Biomaterials. May 2005; 26(14):1857-1875.
23. Evans M J, Kaufman M H. Establishment in culture of pluripotential cells from mouse embryos. Nature. Jul. 9, 1981; 292(5819):154-156.
24. Risau W, Sariola H, Zerwes H G, et al., Vasculogenesis and angiogenesis in embryonic-stem-cell-derived embryoid bodies. Development. March 1988; 102(3):471-478.
25. Hirashima M, Kataoka H, Nishikawa S, et al., Maturation of embryonic stem cells into endothelial cells in an in vitro model of vasculogenesis. Blood. Feb. 15, 1999; 93(4):1253-1263.
26. Yamashita J, Itoh H, Hirashima M, et al., Flk1-positive cells derived from embryonic stem cells serve as vascular progenitors. Nature. Nov. 2, 2000; 408(6808):92-96.
27. Dinsmore J, Ratliff J, Deacon T, et al., Embryonic stem cells differentiated in vitro as a novel source of cells for transplantation. Cell Transplant. March-April 1996; 5(2):131-143.
28. Drab M, Haller H, Bychkov R, et al., From totipotent embryonic stem cells to spontaneously contracting smooth muscle cells: a retinoic acid and db-cAMP in vitro differentiation model. Faseb J. September 1997; 11(11):905-915.
29. Qiu H, Fujimori Y, Kai S, et al., Establishment of mouse embryonic fibroblast cell lines that promote ex vivo expansion of human cord blood CD34+ hematopoietic progenitors. J Hematother Stem Cell Res. February 2003; 12(1):39-46.
30. Radisic M, Park H, Shing H, et al., Functional assembly of engineered myocardium by electrical stimulation of cardiac myocytes cultured on scaffolds. Proc Natl Acad Sci USA. Dec. 28, 2004; 101(52):18129-18134.
31. Radisic M, Yang L, Boublik J, et al., Medium perfusion enables engineering of compact and contractile cardiac tissue. Am J Physiol Heart Circ Physiol. February 2004; 286(2):H507-516.
32. Pedrotty D M, Koh J, Davis B H, et al., Engineering skeletal myoblasts: roles of three-dimensional culture and electrical stimulation. Am J Physiol Heart Circ Physiol. Apr. 1, 2005 2005; 288(4):H1620-1626.
33. Deisseroth K, Singla S, Toda H, et al., Excitation-neurogenesis coupling in adult neural stem/progenitor cells. Neuron. May 27, 2004; 42(4):535-552.
34. McBurney M W, Jones-Villeneuve E M V, Edwards M K S, et al., Control of muscle and neuronal differentiation in a cultured embryonal carcinoma cell line. Nature. 1982; 299(5879):165.
35. Edwards M K, Harris J F, McBurney M W. Induced muscle differentiation in an embryonal carcinoma cell line. Mol Cell Biol. December 1983; 3(12):2280-2286.
36. McBurney M W. P19 embryonal carcinoma cells. Int J Dev Biol. March 1993; 37(1):135-140.
37. Moore J C, Spijker R, Martens A C, et al., A P19C16 GFP reporter line to quantify cardiomyocyte differentiation of stem cells. Int J Dev Biol. February 2004; 48(1):47-55.
38. Choi S C, Yoon J, Shim W J, et al., 5-azacytidine induces cardiac differentiation of P19 embryonic stem cells. Exp Mol. Med. Dec. 31, 2004; 36(6):515-523.
39. Nuccitelli R. Endogenous ionic currents and DC electric fields in multicellular animal tissues. Bioelectromagnetics. 1992; Suppl 1: 147-157.
40. Barbee K A. Role of subcellular shear-stress distributions in endothelial cell mechanotransduction. Ann Biomed Eng. April 2002; 30(4):472-482.
41. Yamamoto K, Takahashi T, Asahara T, et al., Proliferation, differentiation, and tube formation by endothelial progenitor cells in response to shear stress. J Appl Physiol. November 2003; 95(5):2081-2088.
42. Yamamoto K, Sokabe T, Watabe T, et al., Fluid shear stress induces differentiation of Flk-1-positive embryonic stem cells into vascular endothelial cells in vitro. Am J Physiol Heart Circ Physiol. Dec. 2, 2004.
43. Chiu J J, Chen L J, Lee P L, et al., Shear stress inhibits adhesion molecule expression in vascular endothelial cells induced by coculture with smooth muscle cells. Blood Apr. 1, 2003; 101(7):2667-2674.
44. Braddon L G, Karoyli D, Harrison D G, et al., Maintenance of a functional endothelial cell monolayer on a fibroblast/polymer substrate under physiologically relevant shear stress conditions. Tissue Engineering. 2002; 8(4):695-708.
45. Gassmann M, Fandrey J, Bichet S, et al., Oxygen supply and oxygen-dependent gene expression in differentiating embryonic stem cells. Proc Natl Acad Sci USA. Apr. 2, 1996; 93(7):2867-2872.
46. Carmeliet P, Dor Y, Herbert J-M, et al., Role of HIF-1 alpha in hypoxia-mediated apoptosis, cell proliferation and tumour angiogenesis. Nature. Jul. 30, 1998 1998; 394(6692):485.
47. Hirashima M, Ogawa M, Nishikawa S, et al., A chemically defined culture of VEGFR2+ cells derived from embryonic stem cells reveals the role of VEGFR1 in tuning the threshold for VEGF in developing endothelial cells. Blood. Mar. 15, 2003; 101(6):2261-2267.

What is claimed is:

1. Apparatus for culturing and separating cells, comprising:
(a) a bioreactor for growing the cells in cell culture media;
(b) a closed fluid circuit, connecting inlet and outlet portions of the bioreactor, through which cells and media from the bioreactor are circulated;
(c) a pump for pumping media to the bioreactor and for pumping cells and media through the fluid circuit;
(d) an inlet port for introducing magnetic particles into the bioreactor for magnetically labeling cells in culture in the bioreactor; and
(e) a magnetic separator, on the fluid circuit, comprising a controllable magnet, for separating magnetically labeled cells from circulating media within the fluid circuit.

2. The apparatus of claim 1 wherein the magnetic separator further comprises a diverter, responsive to an electromagnet, and a collection chamber, attached to the diverter, wherein labeled cells are separated from unlabelled cells on the basis of magnetic labeling.

3. The apparatus of claim 1 further comprising an optical detector, coupled to the magnetic separator, wherein the electromagnet is controlled in response to detection of an optical signal by the optical detector.

4. The apparatus of claim 1 further comprising a microscope for visualizing cells in the bioreactor.

5. The apparatus of claim 4 further comprising an electrode for delivering electrical pulses to cells adherent to a surface in the bioreactor.

6. The apparatus of claim 1 wherein the bioreactor is adapted for culture of cells in a three dimensional matrix.

7. The apparatus of claim 1 wherein said pump is a pulsatile pump.

8. The apparatus of claim 1, further comprising, in kit form, cell culture media, stem cells, and growth and differentiation factors.

9. A method for separating mammalian cells on the basis of controlled cellular differentiation, comprising the steps of:
(a) continuously growing cells which are not terminally differentiated in a bioreactor having a fluid circuit for media and cells comprising an inlet port to the bioreactor and an outlet port from the bioreactor, said outlet port leading to a magnetic separator;
(b) said growing further being in the presence of factors promoting differentiation into a pre-selected cell type;
(c) labeling the cells with a magnetic particle complex specific for cells not having the pre-selected cell type;
(d) treating cells in the bioreactor to remove a portion of the cells in culture and their media from the bioreactor to the magnetic separator; and
(e) removing undifferentiated cells from the cell culture with the magnetic separator, while returning differentiated cells to the bioreactor via the inlet port for further culture.

10. A method for separating mammalian cells on the basis of controlled cellular differentiation, comprising the steps of:
(a) growing stem cells in a bioreactor having an inlet port for media and cells, and
an outlet port for cells in culture,
said outlet port leading to a magnetic separator;
(b) said growing including multiple cell divisions;
(c) said growing further comprising culturing the cells in the bioreactor under conditions promoting differentiation of the stem cells into a pre-selected cell type;
(d) treating the cells in culture in the bioreactor with a differentiation factor for promoting differentiation into a selected cell type;
(e) labeling the cells in culture with a magnetic particle and an immuno-reactive molecule specific for non-differentiated cells;
(f) treating cells in the bioreactor to remove labeled cells in culture and their media from the bioreactor to the magnetic separator; and
(g) removing undifferentiated cells from the cell culture with the magnetic separator.

11. The process of claim 10 wherein the factors of step (b) and their respective cell type to be produced are as follows:

| Factor | Phenotype |
|---|---|
| electrical stimulation | myocyte |
| media shear | cardiac myocyte, endothelial or fibroblast |
| IGF-1 | myoblast |
| VEGF | cardiac endothelial cells |
| BDNF | cardiac endothelial cells |
| LIF | undifferentiated |
| BMP | undifferentiated |
| PDGF-BB | undifferentiated |
| dibutryl-cyclic AMP | smooth muscle |
| retinoic acid | smooth muscle |

12. The process of claim 11 wherein the immuno-reactive label labels a marker as follows:

| Marker | Phenotype |
|---|---|
| SSEA-1 | undifferentiated cells |
| alkaline phosphatase | undifferentiated cells |
| Flk1 | endothelial or smooth muscle progenitors cells |
| CD31 | endothelial cells |
| Actin | smooth muscle |
| Calponin-h1 | smooth muscle |
| SM Myosin heavy chain | smooth muscle |

13. The method of claim 12 wherein the marker is marked with an antibody bound to a magnetic particle.

14. The method of claim 10 wherein said culturing comprises attaching the cells to beads

15. The method of claim 14 wherein the culturing further comprises introducing the beads into a three dimensional matrix.

16. The method of claim 10 further comprising the step of pumping media through the bioreactor in pulses.

17. The method of claim 10 further comprising administering periodic electrical pulses to the cells.

18. The method of claim 10 further comprising the steps of labeling the cells in culture with an optical label, detecting an optical signal from labeled cells, and magnetically removing cells only which produce an optical signal.

19. The method of claim 10 wherein the separation step is repeated multiple times during a single culture.

20. The process of claim 10 wherein the selected cell type is one of: endothelial cells, smooth muscle cells, and fibroblasts for use in a vascular graft.

21. A method for separating mammalian cells on the basis of controlled cellular differentiation, comprising the steps of:
(a) continuously growing cells which have not terminally differentiated in a bioreactor having an inlet port for media and cells, and an outlet port for cells in culture, said outlet port leading to a magnetic separator;
(b) said growing further being in the presence of factors promoting growth of the cells into a pre-selected cell type;
(c) labeling the cells with a magnetic particle and an immuno-reactive label specific for cells having the pre-selected cell type;
(d) treating cells in the bioreactor to remove a portion of the cells in culture and their media from the bioreactor to the magnetic separator; and
(e) removing differentiated cells from the cell culture with the magnetic separator.

22. Apparatus for culturing electrically responsive cells, comprising:
(a) a bioreactor for growing the cells in cell culture media;
(b) a closed fluid circuit, connecting inlet and outlet portions of the bioreactor, through which cells and media from the bioreactor are circulated;
(c) a pulsatile pump for pumping media in pulses to the bioreactor through an inlet port;
(d) an electrode and electronics for delivering a pulsed electrical field to the cells in the cell culture media; and
(e) a culture surface containing basement membrane material to which the cells adhere.

23. The apparatus of claim 22 wherein the culture surface is attached to a flexible tube connected to the pulsatile pump.

24. The apparatus of claim 23 further comprising a movable mechanism for stretching the tube.

25. The apparatus of claim 23 where the electrode is annular and substantially coaxial with the flexible tube.

26. The apparatus of claim 22 further comprising a magnetic separator.

27. A method for culturing cardiomyocyte cells comprising the steps of:
(a) growing the cells on a basement membrane containing cell culture surface in a bioreactor having a fluid circuit for media and cells comprising an inlet port to the bioreactor and an outlet port from the bioreactor;
(b) applying a pulsed mechanical force to the cells; and
(c) applying a pulsed electrical field to the cells, whereby
(d) said cardiomyocyte cells, after a period in culture, exhibit synchronization.

28. The method of claim 27 wherein the pulsed mechanical force is one or both of (i) pulsed flow of culture media and (ii) stretching of the cell culture surface.

29. The method of claim 27 further comprising the step of culturing the cells in the presence of VEGF.
